SEQUENCE LISTING (18 sequences)

SEQ ID NO: 1 - protein, 868 aa, Virus HIV-1
MAMRAKGIRKNCQHLWRWGTMLLGMLMICSAAANLWVTVYYGVPVWKEATTTLFCASDAKAYDTEAHNVWATHACVPTNPNPQEVVLENVTENFNMWKNNMVEQMHEDIISLWDQSLKPCVKLTPLCVTLNCTDLNTNNTTNTTELSIIVVWEQRGKGEMRNCSFNITTSIRDKVQREYALFYKLDVEPIDDNKNTTNNTKYRLINCNTSVITQACPKVSFEPIPIHYCTPTGFALLKCNDKKFNGTGPCTNVSTVQCTHGIRPVVSTQLLLNGSLAEEEVVIRSENFTNNAKTIIVQLNVSVEINCTRPNNHTRKRVTLGPGRVWYTTGEILGNIRQAHCNISRAQWNNTLQQIATTLREQFGNKTIAFNQSSGGDPEIVMHSFNCGGEFFYCNSTQLFNSAWNVTSNGTWSVTRKQKDTGDIITLPCRIKQIINRWQVVGKAMYALPIKGLIRCSSNITGLLLTRDGGGENQTTEIFRPGGGDMRDNWRSELYKYKVVKIEPLGVAPTKAKRRVVQREKRAVGMLGAMFLGFLGAAGSTMGATSMALTVQARQLLSGIVQQQNNLLRAIKAQQHLLQLTVWGIKQLQARILAVERYLKDQQLLGFWGCSGKLICTTAVPWNASWSNKTLDQIWNNMTWMEWDREIDNYTHLIYTLIEESQNQQEKNQQELLQLDKWASLWTWSDITKWLWYIKIFIMIVGGLIGLRIVFAVLSIVNRVRQGYSPLSFQTLLPNPRGPDRPEGTEEGGGERGRDGSTRLVHGFLALVWDDLRSLCLFSYHRLRDLLLIVARIVELLGRRGWEVLKYWWNLLQYWSQELKNSAVSLVNVTAIAVAEGTDRVIEVVQRIYRAFLHIPRRIRQGFERALL

SEQ ID NO: 2 - protein, 5 aa, Artificial Sequence
  Hydrophobic peptide added to the terminus of the antigenic peptide
FLLAV

SEQ ID NO: 3 - protein, 5 aa, Artificial Sequence
  Hydrophobic peptide added to the terminus of the antigenic peptide
VALLF

SEQ ID NO: 4 - protein, 10 aa, Artificial Sequence
  Hydrophobic decapeptide
GGYCFVALLF

SEQ ID NO: 5 - protein, 68 aa, P. falciparum
NANPNANPNANPNANPNANPNANPNANPNANPNANPNANPNANPNANPNANPNANPNANPNVDPNVDP

SEQ ID NO: 6 - DNA, 20 bp, Artificial Sequence
  Synthetic linker
gatcccgggtgactgactga

SEQ ID NO: 7 - DNA, 20 bp, Artificial Sequence
  Synthetic linker
gatctcagtcagtcacccgg

SEQ ID NO: 8 - protein, 16 aa, Artificial Sequence
  Synthetic oligopeptide
GNVQAAKDGGNTAAGR

SEQ ID NO: 9 - protein, 16 aa, Artificial Sequence
  Trypanosomal peptide pepG
YGGGCTQITEPTCNSS

SEQ ID NO: 10 - protein, 10 aa, Artificial Sequence
  Trypanosomal peptide pepM1
YGVPVATQTG

SEQ ID NO: 11 - protein, 12 aa, Artificial Sequence
  Trypanosomal peptide pepCM1
CYGVPVAQTQTG

SEQ ID NO: 12 - protein, 30 aa, Artificial Sequence
  Trypanosomal peptide pepCM3
CYGVPVAQTQTGVPVAQTQTGVPVAQTQTG

SEQ ID NO: 13 - protein, 47 aa, Artificial Sequence
  Trypanosomal peptide pepM5
YGVPVAQTQTGVPVAQTQTGVPVAQTQTGVPVAQTQTGVPVAQTQTG

SEQ ID NO: 14 - protein, 48 aa, Artificial Sequence
  Trypanosomal peptide pepCM5
CYGVPVAQTQTGVPVAQTQTGVPVAQTQTGVPVAQTQTGVPVAQTQTG

SEQ ID NO: 15 - protein, 7 aa, Artificial Sequence
  Trypanosomal peptide pepL1
KYNATKA

SEQ ID NO: 16 - protein, 8 aa, Artificial Sequence
  Trypanosomal peptide pepCL1
CKYNATKA

SEQ ID NO: 17 - DNA, 34 bp, Artificial Sequence
  Synthetic DNA hydrophobic decapeptide coding sequence
ggtggttactgcttcgttgctctgctgttctgag

SEQ ID NO: 18 - DNA, 38 bp, Artificial Sequence
  Complementary chain of the hydrophobic decapeptide encoding sequence
tcgactcagaacagcagagcaacgaagcagtaaccacc

(19) United States
(12) Patent Application Publication
(10) Pub. No.: US 2002/0155120 A1
(43) Pub. Date: Oct. 24, 2002

(54) Protein and peptide vaccines for inducing mucosal immunity

(76) Inventors: George H. Lowell, Baltimore, MD (US); Thomas C. Vancott, Brookesville, MD (US); Deborah L. Birx, Rockville, MD (US)

Correspondence Address: Karen B. Dow, Morrison & Foerster LLP, Suite 500, 3811 Valley Centre Drive, San Diego, CA, 92130-2332, US

(21) Appl. No.: 09/938,406

(22) Filed: Aug. 21, 2001

Related U.S. Application Data
(62) Division of application No. 09/214,701, filed on Sep. 30, 1999.
Application No. 09/214,701 is a 371 of international application No. PCT/US97/12253, filed on Jul. 10, 1997.
(60) Provisional application No. 60/021,687, filed on Jul. 10, 1996.

(51) Int. Cl. A61K 39/00; A61K 39/12; A61K 39/21; A61K 39/385; C07K 1/00; C07K 14/00; C07K 17/00

(52) U.S. Cl. 424/185.1; 424/186.1; 424/188.1; 424/193.1; 424/196.11; 424/208.1; 530/350

(57) ABSTRACT

A novel vaccine composition combines a protein or peptide antigen, optionally added hydrophobic material and an immunopotentiating membranous carrier which together preserve the antigenic integrity of the protein or peptide epitopes while at the same time enhancing their immunogenicity. Administration of this composition to a subject provokes a protective immune response comprising secretory neutralizing antibodies present in various mucosal sites in the body. This vaccine and the process for using it is intended for use against pathogenic organisms, in particular those causing sexually transmitted diseases or mucosally transmitted diseases. Such organisms include bacteria and enveloped viruses, particularly HIV-1.

### BACKGROUND OF THE INVENTION

1. Field of the Invention

This invention relates to compositions and processes for inducing an immune response against a pathogenic organism such as a causative agent of a sexually-transmitted or mucosally-transmitted disease involving the nasal or respiratory administration of an antigen such as an envelope protein, e.g. an HIV oligomeric gp160, along with proteosomes and/or bioadhesive nanoemulsions.

2. Description of the Background Art

The development of peptide subunit or recombinant protein vaccines to protect against pathogenic microorganisms or malignancies has been impeded by lack of sufficient immunogenicity in the peptide and protein preparations. Often the undesired side effects resulting from exposure to an immunogen must be weighed against the adequacy of its immunogenic properties. The enhancement of immunogenicity of small peptides, protein fragments and polypeptide proteins without increasing undesired side effects from exposure to these agents is an important area of investigation. There exists a paucity of non-toxic and non-pyrogenic carriers and adjuvants for human use. Furthermore, carriers that are safe for human use frequently cannot be efficiently complexed to peptides to render them immunogenic without altering their antigenic structure.

The development of substitutes for whole organism vaccines, or vaccines comprising large proteins therefrom, is an important area of progress in biotechnology. Advances in biotechnology have made it increasingly possible to produce vaccines composed of amino acid sequences identical to protein regions conserved among many strains of given pathogen that may elicit cross-reacting antibodies. If the antigenicity can be improved by coupling proteins or peptides with fragments or other materials that increase immunogenicity, improved vaccines can be developed.

The prior work of several researchers is of interest in this regard. Zollinger et al., (J. Clin. Invest., 63:836-848 (1979)) and Frasch, et al. (In: The Pathogenic Neisseriae, G. Schoolnik, ed., Praeger, N.Y., 1985, pp 633-640) used hydrophobic complexing to make outer membrane protein-polysaccharide vaccines. However, they did not disclose the hydrophobic complexes disclosed herein.

Coon et al (J. Immunol. 110:183-190 (1973)) found that when lauric acid was heavily conjugated covalently to a large protein, bovine serum albumin, humoral immunogenicity was absent, but cell mediated immunity could be induced. Lauric acid was not used to enhance humoral immunogenicity and peptides or protein fragments or hydrophobic complexes were not used.

Hopp (Molec. Immunol., Vol. 21, pp 13-16 (1984)) disclosed the addition of dipalmityl-lysine to a peptide to enhance its immunogenicity. The immunopotentiation reported by Hopp was exceedingly short-lived and induced peak titers that were only system disclosed herein.

Morein and Simons (Vaccine 3:83-93 (1985)) described immunogenic complexes called iscoms between antigenic proteins and glycosides.

The production of immune response to Plasmoduim falciparum, the causative agent of malaria, is of particular interest. World-wide, malaria is the most common serious infectious disease affecting humans. P. falciparum has a tandemly repeated circumsporozoite (CS) tetrapeptide (NANP), which has been the subject of much vaccine research. G. N. Godson, in Molecular Approaches to Malaria Vaccines, discusses the repeated antigenic sequences in the circumsporozoite protein. When an animal is injected with sporozoites most of the antibodies it makes are directed against the CS protein, specifically against the repeating epitope.

European patent publication EPA 191,748 published Aug. 20, 1986 (incorporated herein by reference) refers to an E. coli expression vector having a sequence coding for all or a portion of the repeat unit of the CS protein and discloses a process for purifying the immunogenic polypeptide from the E. coli culture.

European patent publication EPA 192,626, published Aug. 27, 1986 (incorporated herein by reference) refers to an immunogenic polypeptide capable of conferring immunity to P. falciparum infection in mammals which comprises four or more tandem repeat units of the CS protein. The repeat unit is a tetrapeptide system having the sequence Asn Ala Asn Pro (as above).

WO87/06939 (Nov. 19, 1987) teaches a process for isolating and purifying the CS protein expressed in recombinant E. coli.

Dame et al., U.S. Pat. No. 4,707,357, discloses an anti-malarial immunogenic stimulant comprising an immunogenic carrier and a peptide sequence of between 2 and 100 consecutive repeats of a sequence Asn X Y Pro, wherein X is Ala or Val and Y is Asn or Asp. The carriers include soluble molecules such as proteins and polysaccharides and particles such as liposomes and bacterial cells or membranes. The peptide is attached to the carrier by an amide bond formed between a carboxylate or amino groups of a carrier and, respectively, amino or carboxylate group of the peptide. The bonding may be through either an ether or ester linkage. Other disclosed carriers include terminal diamines with 1-10 methylene carbons joining the amines. Preferred carriers were said to be tetanus toxoid and amphiphilic proteins having a lipophilic portion and a hydrophilic portion.

WO86/05790, published Oct. 9, 1986, discloses immunogenic antigen-carrier protein conjugates for use as vaccines against malaria. The conjugates contain the peptide H₂N-(Asn Ala Asn Pro)₃COOH, also designated (NANP)₃. This document also describes a preferred carrier such as tetanus toxoid. Other carriers include diphtheria toxoid and synthetic peptides and polymers comprising lysine and arginine groups. The peptide is coupled to the carrier with the coupling reagent glutaraldehyde or by adding a cysteine residue to the N-terminus and using another conventional ester as a coupling reagent.

Schlessinger, et al., U.S. Pat. No. 4,769,235 (incorporated herein by reference) refers to epitopes having the sequence of an immunodominant epitoper from the repeat region of the CS protein which is shorter in length than the repeating unit. This peptide was active as a vaccine when coupled with a conventional carrier.

WO86/00911 published Feb. 13, 1986 refers to the use of a peptide having the amino acid sequence (Pro Asn Ala Asn)ₙ (where n≧23) adsorbed or coupled to a conventional vaccine carrier protein.

Alum absorbed vaccines containing various forms of the CS epitope have not been sufficiently immunogenic for general human use. Many protein carriers and liposomes known in the art require lipid A or other adjuvants not acceptable for human use.

Human Immunodeficiency Virus (HIV-1) is now well established as the etiologic agent of acquired immunodeficiency syndrome (AIDS). The product of the HIV envelope (env)gene HIV is synthesized as a gp160 precursor molecule which is subsequently processed into the external envelope protein gp120 and the transmembrane protein gp41. The precursor/product relationship between gp160 and its products, gp120 and gp41, as well as the amino acid sequences of all three, are well documented (Allan, et al., Science 228:1091-1094 (1985); Veronese et al, Science, 229:1402-1405 (1985). In addition to their role in cell surface receptor recognition and cell fusion, HIV gp120 and gp41 are the primary targets for immune recognition in HIV-infected subjects. These proteins have therefore received much attention in virus neutralization studies and vaccine development. Large segments of recombinantly expressed gp120 (rgp120), or native gp120 purified from HIV-infected cells, elicit mostly type-specific neutralizing antibodies in animals.

U.S. Pat. No. 5,116,740 and Kalyanaraman, V. S. et al., AIDS Res Hum Retrovir. 4:319-329 (1988) describe the establishment of a process capable of producing the HIV glycoprotein gp160 in its native form. Normally, native gp160 breaks down into gp120 and gp41. Consequently, the envelope protein obtained from cell culture media or from lysed virions is gp120 and gp41. Before the work leading to the above patent, the glycoprotein gp160 had only been produced through recombinant means. Recombinant gp160 is different than the native gp160, particularly in regard to glycosylation, and these differences appeared critical in the search for an HIV vaccine, given that the HIV-1 envelope glycoproteins determine viral tropism and harbor epitopes which are essential for the development of neutralizing antibodies against the virus. U.S. Pat. No. 5,116,740 disclosed a useful clone of HUT78 cells (termed 6D5451) which, when chronically infected with the HIV strain known as “HTLV-III₄₅₁,” released functionally intact viral glycoprotein gp160 in its native form into the culture medium.

Kalyanaraman, V. S. et al., AIDS Res Hum Retrovir. 6:371-380 (1990), described purification of HTLV-III(451) gp120 and gp160 were by sequential affinity chromatographic steps using a mAb to HIV-1 gp41 and an anti-HIV-1-positive human serum. Amino acid sequence analysis of gp120 and gp106 showed the loss of the signal peptide. Both proteins were heavily glycosylated and contained complex carbohydrates, in contrast to the intracellular form of gp160 which had been shown to contain mannose-rich immature sugars. The affinity of gp160 was five times lower than that of gp120 for binding CD4. Both gp120 and gp160 inhibited syncytia formation by HIV-1-infected cells when mixed with CD4+ cells. Both gp120 and gp160 had strong mitogenic effects on T cells from HIV-1-infected gibbons but not on cells from uninfected gibbons.

U.S. Pat. No. 5,116,740 (supra) and VanCott T. C et al., J. Immunol. Meth. 183:103-117 (1995), assessed the oligomeric structure and antigenic properties of an affinity purified gp160 protein (oligo-gp160 or “o-gp160”) using biosensor technology and identified the existence of tetrameric, dimeric and monomeric forms of the protein. (VanCott and co-author Birx are named co-inventors in this application.) Monoclonal antibodies specific for oligomeric gp160 reacted with discontinuous epitopes within monomeric gp120 and several linear epitopes within gp120 (V3) and gp41. Sera from HIV-infected subjects from around the world, including places where HIV-1 subtypes A-F and O (African) were reactive with oligo-gp160. This indicated the preservation of conserved antigenic epitopes in this material. Furthermore, enhanced immunologic reactivity per gp160 molecule was obtained with oligo-gp160 as compared to other current HIV-1IIIB subunit monomeric envelope gp120/gp160 immunogens, leading the authors to conclude that this material had higher HIV-1 envelope protein mimicry. Atni-HIV-1 serum antibodies during acute infection could be detected by oligo-gp160 prior to their detectability with either a recombinant, monomeric gp120 protein or several commercial HIV-1 screening kits. The authors concluded that the oligomeric nature of this gp160 protein preparation and its high reactivity with divergent mAbs and HIV-1 sera support its use as an HIV-1 immunogen.

The foregoing Kalyanaraman et al publications, the VanCott et al paper and U.S. Pat. No. 5,116,740 did not disclose the significance of hydrophobic peptide portions of o-gp160 nor its complexing with proteosomes or nanoemulsions as is disclosed and claimed herein.

Yang, C et al., Proc Natl Acad Sci USA, 92:9871-9875 (1995), disclosed that envelope proteins HIV and simian immunodeficiency virus (SIV) were modified by fatty acylation of the transmembrane protein subunit gp41. The precursor gp160 was also palmitoylated prior to its cleavage into the gp120 and gp41 subunits. The linkage was said to be through a thioester bond. Palmitoylation was a posttranslation modification. In contrast to other glycoproteins, which are palmitoylated at cysteine residues within or close to the membrane-spanning hydrophobic domain, the palmitoylation of the HIV-1 envelope proteins occurs on two cysteine residues, Cys-764 and Cys-837, which are 59 and 132 amino acids, respectively, from the proposed membrane-spanning domain of gp41. One of these residues (Cys-764) was conserved in the cytoplasmic domains of almost all HIV-1 isolates and is located very close to an amphipathic region which has been postulated to bind to the plasma membrane.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present inventors have discovered a novel vaccine composition which combines a protein or peptide antigen, optionally added hydrophobic material and an immunopotentiating membranous carrier which together preserve the antigenic integrity of the protein or peptide epitopes while at the same time enhancing their immunogenicity. Administration of this composition to a subject provokes a protective immune response comprising secretory neutralizing antibodies present in various mucosal sites in the body. This vaccine and the process for using it is preferably intended for use against pathogenic organisms, in particular those causing sexually transmitted diseases or mucosally transmitted diseases, including diseases spread by non-sexual spread through body fluids or mucosal secretions, including fecal spread. Such organisms include bacteria and enveloped viruses, particularly HIV-1.

In one embodiment, the present inventions provides a vaccine composition capable of eliciting neutralizing antibodies in a subject to a pathogenic organism which antibodies are present in vaginal secretions, intestinal secretions, lung secretions or feces, which composition comprises:

(a) an antigen comprising a protein or peptide having

(i) an endogenous hydrophobic sequence of between about 3 and about 50 non-polar or uncharged amino acids;

(ii) added to the protein or peptide, an exogenous hydrophobic material comprising a sequence of between about 3 and about 50 non-polar or uncharged amino acids or a C8-C 18 fatty acyl group; or

(iii) both (i) and (ii),

(b) complexed with the antigen, a composition comprising proteosomes, bioadhesive nanoemulsions, or both, wherein the complexed or coupled protein or peptide maintains a native structure of antigenic epitopes such that, upon administration to the subject, the antigen induces neutralizing antibodies in one or more of vaginal secretions, intestinal secretions, lung secretions and feces, capable of neutralizing the pathogenic organism.

The endogenous hydrophobic sequence or the exogenous hydrophobic sequence is an amino acid sequence is preferably between about 5 and about 29 residues. Preferred short exogenous hydrophobic sequences are Phe-Leu-Leu-Ala-Val or Val-Ala-Leu-Leu-Phe. The exogenous hydrophobic material may also be C8-C18 fatty acyl group, preferably lauroyl.

A preferred protein is a viral envelope protein, such as oligomeric gp160 from HIV-1. In one embodiment, the gp160 has the sequence of residues 33-681 of SEQ ID NO:1.

The antigen may be a peptide or a peptide oligomer. In one embodiment, the protein or peptide naturally contains or has added to it at least one cysteine residue.

The protein or peptide is chemically synthesized or recombinantly produced.

In the above vaccine, the antigenic protein is complexed with proteosomes which are preferably hydrophobic, multimolecular membrane proteins. The vaccine composition is preferably formed by: (a) bonding the hydrophobic material to the protein or peptide to form a hydrophobic-hydrophilic compound; and (b) admixing the compound with the proteosomes, bioadhesive nanoemulsions, or both such that the antigen is complexed with the proteosomes or nanoemulsion. The admixing step is may be performed in the presence of a detergent, and is followed by the step of removing the detergent by dialysis. Alternatively the admixing step is performed lyophilization.

The vaccine composition preferably is formulated for intranasal or respiratory administration, and preferably in a dosage form suitable for multiple inoculations.

Also provided are processes for inducing a neutralizing antibody response in a subject against a pathogenic organism resulting in neutralizing antibodies in one or more of vaginal secretions, intestinal secretions, lung secretions and feces, which process comprises administering to the subject an effective amount of any of the vaccine compositions as described above.

The compositions and processes are particularly useful for inducing protective, neutralizing immunity to a pathogenic organism such as a causative agent of a mucosally transmitted or sexually transmitted disease.

Administration of the vaccine may be prior to or after exposure (or both prior to and after exposure) to the pathogenic organism.

The components of the vaccine composition can by complexed by any means known in the art. Any synthetic or cloned peptide which is to serve as the antigen can have a hydrophobic foot and/or a Cys added. Therefore any peptide can be made immunogenic by this approach. Hence this method differs from chemical covalent coupling of the prior art which depends on the correct chemistry to attach and orient a peptide epitope appropriately.

The proteosomes used in the vaccine composition are preferably hydrophobic multimolecular membrane proteins. They may be obtained from any of a number of different organisms; in one embodiment, they are derived from meningococci. Complexing of the proteosomes with the antigenic component may be accomplished by any of a number of means, preferably by dialysis or lyophilization as noted above.

### BRIEF DESCRIPTION OF THE DRAWINGS

FIG. 1 is a schematic drawing illustrating a schedule of immunization and sample collection. The following abbreviations are used: SE—serum; VG—vaginal wash; FE—fecal pellets; LG—lung lavage; IN—intestinal lavage; SPL,—spleen.

FIGS. 2A, 2B and 2C are a series of graphs showing the effects of intranasal immunization with oligo gp-160 formulated with proteosomes and/or bioadhesive nanoemulsions. Neutralizing antibodies were found in serum and vaginal and lung fluids and are shown in relative units. These antibodies recognize natively folded gp-120. Antibodies from serum (FIG. 2A), vaginal wash (FIG. 2B) and lung wash (FIG. 2C) were evaluated for their binding to native and denatured gp-120_{MN} using a solid phase radioimmunoassay (SPR). Sera and mucosal washes from mice immunized with oligo gp-160 wither subcutaneous (S.C.) or intranasal (I.N.) preferentially bind native gp-120. Pooled HIV-1 sera known to bind preferentially to native gp-120 served as a positive control. Sera from a rabbit immunized oligo gp-160₄₅₁ in Freund's adjuvant (R265), Ras3C is a lipid A containing adjuvant from RIBI; rgp120MN (recombinant gp120 from virus strain MN) and rcmgp120MN (reduced, carboxymethylated rgp120MN) were from Genentech. Recombinant gp160 from MicroGenesys Mrgp-160:is not natively folded and therefore has much poorer expression of the native epitopes compared to oligo gp160, was another negative control.

FIGS. 3A and 3B are graphs showing the induction of neutralizing antibodies in vaginal wash (VG) (FIG. 3A) and in lung wash (LG) (FIG. 3B) with various of the antigenic preparations indicated. The symbols are as follows: —□—, ---▪--- and —◯—: saline controls; ---●--- o-gp160/proteosomes; —Δ— and ---◯--- : o-gp160/proteosomes-pmax (nanoemulsion).

FIG. 4A, 4B and 4C are graphs showing the induction of HIV-neutralizing antibodies in mice after intranasal immunization with o-gp 160. HIV-1_{MN} neutralizing activity of sera (FIG. 4A), vaginal wash (FIG. 4B) and lung wash (FIG. 4C) are shown from mice immunized subcutaneously (s.c.) or intranasally (i.n.) with o-gp160₄₅₁ formulated in proteosomes (“prot”), “emulsomes” (“emul”) or proteosome+emulsomes (“prot/emul”). Sera or mucosal washes from 5 mice per group were pooled. Results are shown as the mean and standard deviation of 3-5 replicate wells for each dilution of serum or mucosal wash. Pre-immunization (“pre”)) and post-immunization (“post”) serum pools in both the i.n. and s.c. immunized groups are shown for comparison. Positive control sera in FIG. 4A were strongly (US9) and moderately (US18) neutralizing HIV-1 patient sera. Vaginal and lung washes from mice receiving saline only were negative controls in FIG. 4B and 4C, respectively. The results are indicated as follows: FIG. 4A: ---▪--- s.c.-Ras3C-pre; —□— s.c.-Ras3C-post (Ras3C is a commercial semisynthetic adjuvant); ---●--- in.-prot-prep; —◯— i.n.-prot-post; ---▴--- i.n.-prot/emul-pre; —Δ— i.n.-prot/emul-post; --- [symbol] --- US18; FIG. 4B: ---▪--- i.n.-saline, ---▴--- i.n.-saline; ---●--- i.n.-saline;, —□— i.n.-prot; —Δ— i.n.-prot/emul; —◯— i.n.-prot/emul FIG. 4C: ---▪--- i.n.-saline; ---●--- i.n.-saline; ---▴--- i.n.-saline; —□— i.n.-prot, —◯— i.n.-prot; —Δ— i.n.-prot/emul; --- [symbol] --- i.n.-prot/emul

### DESCRIPTION OF THE PREFERRED EMBODIMENTS

A suitable immunopotentiating system suitable for use with this invention renders peptides (including small peptides) immunogenic and enhances the immunostimulating properties of larger peptides, proteins, and protein fragments. As generally understood, “immunostimulating” is defined as the capacity to induce a T cell-mediated immune response, such as delayed hypersensitivity or cytotoxic T lymphocytes, and/or an antibody response. The desired amino acid sequences which comprise the epitope(s) to which immunity is desired, may be made by chemical synthesis from amino acids and/or polymerization, by extraction from the pathogen, or by recombinant means. The antigenic peptides or proteins used in the vaccine may vary in sequence from the native antigenic or immunogenic sequences of the natural pathogens by addition, deletion, or insertion of other amino acids or by the attachment of additional sequences, preferably hydrophobic moieties. The antigenic peptide itself may be positively or negatively charged or neutral.

The exogenous hydrophobic material, also termed the “hydrophobic foot” or “hydrophobic anchor,” is optionally attached to the antigenic, immunostimulating sequence and may vary in structure. A preferred hydrophobic foot comprises a fatty acyl group containing from about 8 to 18 carbon atoms and bonded in an amide bond to the protein or peptide. In a preferred embodiment the group is an alkanoyl group. A particularly preferred alkanoyl group is a lauroyl group. Hydrophobic groups of this type are easily added to the amino terminus of a synthetic peptide while the peptide is still on the resin used for synthesis. Peptides may be synthesized by the solid phase method (Merrifield, B., J Amer. Chem. Soc. 85:2149-2154 (1963); Merrifield, B., Science 232:341-347 (1986); Wade, J. D. et al., Biopolymers 25:S21-S37 (1986); Fields, G. B., Int. J. Peptide Prot. Res. 35:161 (1990); MilliGen Report Nos. 2 and 2a, Millipore Corporation, Bedford, Mass., 1987). When a synthetic peptide is used, the fatty acyl group preferably as an alkanoyl chloride, can be reacted with the peptide on the resin.

The fatty acyl group may also be added to the amino terminus by reaction of a fatty acid such as lauric acid. To avoid side reactions, free amino groups may be blocked to assure that the acyl group is attached to the end of the peptide. It is also possible to attach the acyl group on the carboxy terminus using Lys as the C-terminal amino acid and reacting the acyl group with the epsilon amino group of Lys by conventional means.

The exogenous hydrophobic anchor may also be a hydrophobic peptide of about 3 to 50 amino acids, preferably between about 5 and 24 amino acids in length. Such a hydrophobic peptide preferably consists of non-polar or neutral amino acids, although, the longer this peptide, the greater the tolerance for up to several charged amino acid residues. The amino acids that are particularly useful in providing hydrophobicity and are preferred for the hydrophobic peptide sequence of this invention are drawn from the following categories:

1. Small aliphatic, nonpolar residues: Ala, Thr or Pro

2. Large aliphatic, nonpolar residues: Met, Leu, Ile, or Val ; and

3. Large aromatic residues: Phe, Tyr, Trp.

Hydrophobic amino acids of longer chain length can also serve the function of the hydrophobic foot so long as the length of the hydrophobic foot does not exceed about 24-30 amino acids. It is important that the antigenic peptide bound to an exogenous hydrophobic foot not be rendered totally water insoluble in the presence of detergent.

A specific example of endogenous hydrophobic regions associated with HIV gp160 is the transmembrane domain of the gp41 portion of gp160. This region has a membrane spanning hydrophobic domain as well as other hydrophobic sequences, for example, amino acids 523-551 of the gp160 sequence shown below (SEQ ID NO:1). Such a sequence may be naturally rendered even more hydrophobic by palmitoylation (Yang et al., supra). Thus, for example in SEQ ID NO:1, the Cys at position 776 would be palmitoylated (corresponding to position 764 in Yang et al.). This is a natural case of an embodiment of this invention wherein a fatty acyl group is added to an antigenic peptide and serves as the hydrophobic foot. Native gp160 appears to have several of its own hydrophobic feet, in the form of hydrophobic amino acid domains as well as the more recently discovered palmitoylated sites, which benefit its utility in the present vaccine compositions and methods.

In the instance where the antigenic component of the vaccine is a peptide synthesized by sequential solid or liquid phase synthesis, the hydrophobic peptide sequence may be added synthetically or recombinantly to either terminus of the antigenic peptide. A preferred hydrophobic peptide is a pentapeptide, most preferably Phe-Leu-Leu-Ala-Val or Val-Ala-Leu-Leu-Phe (SEQ ID NO: 2 and 3].

Immunogenicity of longer peptides and proteins may also be potentiated by methods disclosed herein. Many extracted or cloned polypeptides (especially transmembrane polypeptides) have natural hydrophobic anchors which are frequently 15 to 30 amino acids long. The immunogenicity of such polypeptides may also be enhanced by extending a native hydrophobic anchor or by adding another hydrophobic anchor according the methods disclosed herein. A preferred decapeptide is Gly-Gly-Tyr-Cys-Phe-Val-Ala-Leu-Leu-Phe (SEQ ID NO:4) because it has appropriate size and composition to allow easy purification of a recombinant anchored protein. Native sequences may have length and composition which hinder extraction and purification.

The hydrophobic anchor sequence is preferably added to the carboxy-terminus of the selected recombinant protein by genetic engineering methods. Hence, the polynucleotide that encodes the anchor can be added to the 3′ end of the coding sequence for the desired recombinant protein. Alternatively, the polynucleotide that encodes the anchor may also be added at the 5′ end of the selected coding sequence. In another embodiment, the polynucleotide that encodes the anchor may be added to both the 5′ and 3′ termini of the sequence encoding the selected protein. For conventional techniques to accomplish construction of these vectors, see, for example, Sambrook, J. et al., Molecular Cloning: A Laboratory Manual, 2nd Edition, Cold Spring Harbor Press, Cold Spring Harbor, N.Y., 1989; Ausubel, F. M. et al. Current Protocols in Molecular Biology, Wiley-Interscience, New York, 1987 (or most recent volume).

The constructs can be complexed to the proteosomes by dialysis or lyophilization as described above in methods for preparation with peptides. Similarly, the hydrophobic foot may be attached by the methods indicated for attachment to peptides as an alternative to production in a recombinant molecule as described above.

Ratios of proteosomes to anchored recombinant protein (weight:weight) may ranges between about 1:1 to about 1:20. Preferred ratios are between about 1:1 and about 1:3 for polypeptides or proteins.

The complexing between the proteosomes or nanoemulsions and the antigenic protein or peptide is intended to be non-covalent. Although it is often referred to as hydrophobic, it includes ionic interactions and hydrogen bonding. Since hydrophobic complexing is more physical than chemical, and since hydrophilic protein epitopes remain conserved, exposed, and typically unaltered, antibodies generated against these epitopes will recognize the native protein or an epitope thereof and will therefore function in binding, attacking or removing the pathogen from which the protein or epitope is derived or with which it cross-reacts.

Vaccine compositions may be introduced into a subject by conventional means, including parenteral routes (for example, subcutaneous, intradermal, intramuscular) and by direct application to mucous membranes. Lyophilized compositions may be “snorted” into the nasal cavity. Dosage will depend on the particular agent administered, and will be readily apparent to those skilled in the art.

Epitope Repetition

An oligomeric peptide may be synthesized as a repeating unit wherein the unit peptide sequence is repeated in a tandem array up to as many times as synthesis will allow. Tandem repeating units of 2-6 have been used with enhancing effects on the immune response. Epitope repetition enhances the immunogenicity of the peptide. Complexing the oligomeric peptide with an exogenous hydrophobic material prepared as the methodology described herein, can render a totally non-immunogenic peptide immunogenic without a need for added adjuvants, and in some case, even without a need for proteosomes as described herein. However, complexing the oligomeric peptides directly with proteosomes is also a preferred embodiment. A composition comprising an oligomeric peptide and at least one Cys residue which is complexed with proteosomes is a most preferred vaccine.

Because complexing depends upon the presence of hydrophobic sites in the protein or peptide, the number of peptide molecules that can be complexed to the proteosomes is far greater than the number that can be complexed by ordinary covalent bonding systems. Each proteosome can be complexed with between about 6 and −30 protein or peptide molecules.

Preparation of Immunogenic Peptide Vaccines

Either dialysis or lyophilization may be used to prepare the vaccine as follows:

1. Dialysis Method

a. Combination of Components in TEEN-1%

The proteosomes are stored in TEEN-1% buffer (0.05M Tris-HCl, 0.15M NaCl, 0.01M EDTA (ethylenediamine tetraacetate) and 1% Empigen-BB® (Albricht and Wilson, Cumbria, England) at a concentration ≧1 mg/nil (preferably 1.5-2.5 mg/ml). The proteosome material mixed with the antigenic peptide which is also in a TEEN-1% solution. The peptide may have an endogenous or exogenous hydrophobic foot (with or without an added Cys residue and with or without tandemly repeating epitope, as desired) in a beaker or test tube. Ratios of proteosomal protein to antigenic peptide (weight:weight) that have been used have ranged from 1:1 to 1:40. The usual ratio is 1:1 although, depending on the circumstances, 1:4 or higher may be preferable. The concentration of the peptide in solution prior to admixture with proteosomes must be high enough so that the concentration of both the peptide and the proteosomal protein in the combined mixture is ≧1 mg/ml when the components are at equal ratios. When the ratio is not 1:1, the concentration of the less concentrated component should be ≧0.50 mg/ml and preferably, ≧0.75 mg/ml. For example, if the proteosomes are at 1.1 mg protein/ml, the peptide should be at 10 mg/ml prior to combining at a 1:1 ratio. While these minimal concentrations are not absolute, and although successful vaccines have been prepared using lower protein concentrations (when the peptide:protein ratio is significantly >1:1), the method described above is more consistently successful.

b. Dialysis of the Mixture

The mixture is transferred to dialysis bags that, due to their low molecular weight cutoff, retain both the peptide and the protein while allowing the detergent (usually Empigen-BB) in the TEEN-1% to dialyze away. For this reason, Spectra-Por 6 (or 7) dialysis tubing with a molecular weight cutoff of 1000 is routinely used to maximize the amount of peptide retained for complexing to the proteosomes. The dialysis tubing (closed using special Spectra-Por closures) is washed just prior to use with pyrogen-free distilled water and then phosphate buffered saline pH 8.5 (PBS-8.5) which consists of 0.025 M Na₂HPO₄ plus 0.15 M NaCl (normal saline). The proteosome-peptide mixture is exhaustively dialyzed against this buffer, e.g. at a ratio of 200-250:1 for 10 days with daily changing the dialysis fluid). On the final day of dialysis, the buffer is changed to standard phosphate buffered saline, PBS (Na₂HPO₄+NaH₂PO₄+NaCl at pH 7.4). Under certain circumstances, the dialysis period may be shortened, for example to 5 days with 2 changes of fluid per day.

c. Vaccine Collection

Solution is collected from dialysis bag(s), and the bags are washed with 20% of their volume of PBS. The rinse is combined with vaccine. The vaccine is filtered through a 0.22 μm filter (the vaccine may need to be pre-filtered through a 0.8 or 0.45 μm filter) or through a 0.45 μm filter. The protein content is measured (e.g. by absorbance at 280 nm or by the Lowry assay), and samples taken for amino acid analysis, HPLC and other analyses,

The samples are then diluted in PBS to a concentration of 0.4 mg protein per ml solution. The final vaccine is then diluted 1:1 with either normal saline (supplemented with 0.02% merthiolate as an antibacterial) to yield a 0.2 mg/ml solution. This solution may then be administered intramuscular (i.m.) at 0.5 ml per dose.

Alternatively, if desired, the vaccine can be adsorbed to alum by diluting 1:1 with a solution of alum instead of saline, incubation at room temperature for 2 hrs. with occasional stirring and then incubation at 4° C. for 3-18 hrs. It should be emphasized that the results obtained by the present inventors indicate that the vaccine works perfectly well without alum. One reason for adding alum is to evaluate its role in the long term human response.

2. Lyophilization Method

Instead of combining the proteosomes and antigenic peptide (optionally bonded to a hydrophobic foot) in TEEN-1%, the peptide and proteosomes may be complexed and rendered immunogenic by simply lyophilizing these components together according to the following procedure.

Proteosomes are removed from TEEN-1% by precipitation through the addition of three volumes of 100% ethanol to one volume of the proteosomes, allowing to stand at 4° C. for one hour and then centrifuging them at 800-1000 g for 15 minutes; washing the proteosomes three times by adding the same amount of 100% ethanol as previously used and re-centrifuging as before, then resuspending the proteosomes in PBS to a concentration of 2 mg/ml.

The peptide (with its hydrophobic foot and, if desired, Cys and replicated epitopes) is then redissolved in PBS at 2 mg/ml (or another concentration as described above if a peptide:protein ratio >1:1 is desired). The dissolved peptide is added to the proteosome suspension and mixed. The mixture is lyophilized and, following lyophilization, resuspended to 1 mg protein/ml using distilled water. The product is then filtered, analyzed, diluted and added to saline or alum as described above.

Creation of Peptide Oligomers

The antigenic peptides may be in the form of “peptide oligomers” which may be tandem or cross-linked oligomeric peptides wherein (1) the basic peptide unit is tandemly repeated a number of times, (2) the basic peptide unit contains at least one Cys residue (naturally or by addition), and two or more peptide units are cross-linked by disulfide linkages between Cys residues or (3) two or more basic peptide units are chemically cross-linked.

Epitopes may be repeated in tandem as many times as synthesis will allow. Such structure enhances the immunogenicity of a peptide epitope. When prepared with the methodology described below, a totally non-immunogenic peptide can be made immunogenic without added adjuvants and even without the proteosomes. However, complexing the peptide oligomers with proteosomes is preferred. Epitope repetition may be used in conjunction with the addition of Cys (see below) in the process in which an exogenous hydrophobic foot and this modified peptide is optionally complexed with proteosomes. In either case, toxicity and side effects are minimal.

Addition of Cysteine Residue(s)

The peptides and proteins used in the present compositions and methods may natively contain Cys residues or have Cys residues added as described. Thus, the native presence of Cys in the antigenic peptide or protein is not required.

When the peptides are synthetic, Cys residues may be added during the synthesis. Cys may also be added covalently to previously synthesized sequences by carbodiimide coupling. The Cys is useful for effecting dimerization, oligomerization (if more than one Cys is present) or cyclization of the peptides. Unless reducing agents are present, dimerization occurs spontaneously following deblocking and cleavage of the peptide when one Cys is present in the peptide. In a preferred embodiment one Cys is located between the hydrophobic foot and the antigenic peptide epitope. When the peptide contains two Cys residues, cyclization is accomplished spontaneously in dilute solution after de-blocking and cleavage of the peptide. Ferricyanide oxidation of the peptide in the dilute solution causes formation intrachain (but not interchain) disulfide bonds.

A Cys residue can be added to provide for dimerization (or oligomerization) of either the hydrophilic antigenic epitope, the endogenous hydrophobic sequence or the exogenous hydrophobic group. Dimerization appears to stabilize binding to the proteosome by providing two hydrophobic feet for the epitope. The dimerized constructs also provide for more stable interaction with the antigen. The Cys may be placed at either the C- or N-terminus of the antigenic peptide.

When the antigenic protein or peptide is produced by genetic engineering means, the nucleotide sequence encoding a hydrophobic foot peptide or any desired Cys residue may be attached in-frame to the nucleotide encoding the antigenic protein or peptide.

When Cys is added, it is preferably done as part of the process of forming the vaccine compositions during the step of adding an exogenous hydrophobic maternal (such as during chemical synthesis or recombinant production). In another embodiment, the Cys is added to antigenic peptide that has its endogenous hydrophobic peptide. Two general approaches may be used.

a. Dimerization

Adding one Cys provides a point for dimerization of the hydrophobic foot of this invention and the hydrophilic antigenic peptide. Cys addition allows the enhancement of immunogenicity of an antigenic peptide when used in conjunction with (i) the hydrophobic foot plus proteosomes, (ii) tandemly repeated epitopes or (iii) both of the foregoing.

Without being bound to any particular mechanism, the present inventors conclude that dimerization provides two hydrophobic feet for the epitope, to provide more stable binding to the proteosomes or promote formation of auto-micelles. Furthermore, at least two epitopes created by the dimerization may improve conformation of the peptide epitope and allow a more stable interaction with antigen recognizing cells in the treated subject.

b. Cyclization

Two Cys residues are added to either end of a hydrophilic antigenic peptide, one Cys between the peptide and the hydrophobic foot and one Cys at the distal end of the hydrophilic epitope. After addition of the hydrophobic foot, the peptide may be deblocked and cyclized using an oxidizing agent such as ferricyanide. The chemistry of these reactions is well-known in the art.

HIV-1 Envelope Glycoproteins for Use in Vaccine Composition

A preferred antigenic protein for use herein is an envelope protein of HV-1. The mature envelope proteins in virions and HIV-infected cells are gp1 20 and gp41, which are derived from a single precursor, gp160. The advantages of a gp160 protein having antigenic epitopes in a native or undamaged form is important for a useful vaccine. This has been discussed above (See: U.S. Pat. No. 5,116,740; VanCott T. C et al., J Immunol. Meth. 1995, 183:103-117). A preferred form of the gp160 is oligo-gp160 (or o-gp160), as disclosed in these references, because of its maintenance of antigenic epitopes, presumably in native-like form, and due to the presence of gp160 dimeric and tetrameric structures in this preparation.

Depending on the isolate or clone used, the o-gp160 protein preparation can have a lower molecular weight. For example in the 451 isolate described in detail herein, the gp160 monomer appears to have a molecular mass of about 140 kDa due to a truncation which had not previously been recognized. Such a lower molecular mass truncation variant could be referred to as “gp140” or “o-gp140” due to its apparent molecular mass of about 140 kDa rather than 160 kDa.

The compositions of the present invention include variants gp160, whether they be amino acid substitution variants (either natural isolates or genetically engineered variants) as well as truncation variants which may have occurred inadvertently (as is believed to be the case for the 451 isolate) or have been deliberately prepared for any of a number of reasons, including improved secretion from cells. Thus, as used herein, the term “gp160” is intended to encompass the disclosed truncation variant and other presently known or later discovered truncation variants and amino acid substitution variants of gp160.

In a preferred embodiment described below the o-gp160 was obtained from the HIV-1 isolate originally named HTLV-III₄₅₁. This protein is listed on the SWISS-PROT database, (maintained by the National Center for Biotechnology Information of the National Institutes of Health, Bethesda, Md.) as Seq ID: 119434, and was shown to have the amino acid sequence shown below (in single letter code).

This sequence (SEQ ID NO:1) is divided as follows: residues 1-32 are the signal peptide ending with the “/” mark. Residues 33-522 constitute gp120, ending with the “\” mark. Residues 523-868 constitute gp41. It was subsequently discovered that this clone was truncated, with the C-terminal 187 amino acids of gp41 missing. These are indicated by underscoring in the sequence below. Thus, the o-gp160 protein as obtained from the cloned cell line described below has only 649 residues (from position 33 to 681 of SEQ ID NO:1).

It is noteworthy that a large hydrophobic region of gp160 is retained in this protein and is indicated in the above sequence in italic and boldface and double underscore. This 29 mer (from positions 523 to 551) is an example of an endogenous hydrophobic sequence and can be exploited in the vaccine composition.

| | |
|---|---|
| 1 | MAMRAKGIRK NCQHLWRWGT MLLGMLMICS AA/ANLWVTVY YGVPVWKEAT |
| 51 | TTLFCASDAK AYDTEAHNVW ATHACVPTNP NPQEVVLENV TENFNMWKNN |
| 101 | MVEQMHEDII SLWDQSLKPC VKLTPLCVTL NCTDLNTNNT TNTTELSIIV |
| 151 | VWEQRGKGEM RNCSFNITTS IRDKVQREYA LFYKLDVEPI DDNKNTTNNT |
| 201 | KYRLINCNTS VITQACPKVS FEPIPIHYCT PTGFALLKCN DKKFNGTGPC |
| 251 | TNVSTVQCTH GIRPVVSTQL LLNGSLAEEE VVIRSENFTN NAKTIIVQLN |
| 301 | VSVEINCTRP NNHTRKRVTL GPGRVWYTTG EILGNIRQAH CNISRAQWNN |
| 351 | TLQQIATTLR EQFGNKTIAF NQSSGGDPEI VMHSFNCGGE FEYCNSTQLF |
| 401 | NSAWNVTSNG TWSVTRKQKD TGDIITLPCR IKQIINRWQV VGKAMYALPI |
| 451 | KGLIRCSSNI TGLLLTRDGG GENQTTEIFR PGGGDMRDNW RSELYKYKVV |
| 501 | KIEPLGVAPT KAKRRVVQRE KR\AVGMLGAM FLGFLGAAGS TMGATSMALT |
| 551 | VQARQLLSGI VQQQNNLLRA IKAQQHLLQL TVWGIKQLQA RILAVERYLK |
| 601 | DQQLLGFWGC SGKLICTTAV PWNASWSNKT LDQIWNNMTW MEWDREIDNY |
| 651 | THLIYTLIEE SQNQQEKNQQ ELLQLDKWAS LWTWSDITKW LWYIKIFIMI |
| 701 | VGGLIGLRIV FAVLSIVNRV RQGYSPLSFQ TLLPNPRGPD RPEGTEEGGG |
| 751 | ERGRDGSTRL VHGFLALVWD DLRSLCLFSY HRLRDLLLIV ARIVELLGRR |
| 801 | GWEVLKYWWN LLQYWSQELK NSAVSLVNVT AIAVAEGTDR VIEVVQRIYR |
| 818 | AFLHIPRRIR QGFERALL |

Cell Culture and Production of oligo-gp160

A single cell clone of HUT78 cells has been infected with human immunodeficiency virus type 1 (HIV-1), resulting in a cell line which continuously produces virus. Clone 6D5 is susceptible to chronic infection with HIV-1, as described in Getchell, et al., J. Clin. Microbiol. 23:737-742 (1986). Clone 6D5 is infected with a specific strain of HIV-1, HTLV-III₄₅₁, to produce the infected cell line 6D5451 (deposited with the American Type Culture Collection under the Budapest Treaty). The infected cell line is then grown in serum-free medium, by pelleting 6D5451 cells and resuspending them in serum-free medium (such as HB101 or HB104 medium, commercially available from Du Pont). The medium also contains growth supplements such as transferrin, insulin, and bovine serum albumin. To assist in the growth of cells, the cells were subcultured every four day. The 6D541 cells were grown for 2 to 3 generations. When serum-free medium is used, glycoprotein gp160 can be separated from other proteins in the medium.

The amount of HIV proteins released into the media, as measured by extracellular reverse transcriptase activity (Poiesz et al., Proc. Natl. Acad Sci. USA, 77:7415-7419 (1980), was nearly five-fold greater in serum-free medium than in serum-containing medium. The cell-free medium is used as the source of the glycoprotein. The medium is adjusted to 20 mM with sodium phosphate, pH 7.5, 0.5% Triton X-100, 0.1 mM phenylmethylsulfonyl fluoride, and 400 mM sodium chloride. After incubation at room temperature for one hour, the medium was concentrated 30-fold with a Pellicon cassette system, commercially available from Millipore. Extraneous proteins derived from the media supplement are removed from the concentrate by immunoaffinity absorption (overnight) with a Sepharose-bound goat antibody raised against the proteins in the growth supplement in the serum-free medium. Proteins which bind to the goat antibody are removed, and the unbound material is passed through a lectin affinity column (preferably lectin-Sepharose®). Although the use of a lentil lectin column is preferred, other lectins which will recognize mannose, such as concanavalin-A, may also be employed. After washing with phosphate buffered saline (PBS), the column is eluted with 400 mM α-methylmannoside to recover the viral glycoprotein. (Any mannose, pyranoside, or saccharide which competes with the lectin in the affinity column may be used.).

Immunoblot analysis of the HIV glycoprotein is carried out by well known procedures, (e.g., Sarngadharan, et al., Science 224:506-508 (1984). Essentially, the proteins are run on 7% SDS-polyacrylamide gels and transferred to commercially available nitrocellulose strips. The strips are then treated with the appropriate antibodies, and the blots are developed with enzyme-conjugated secondary antibodies; the bands are visualized by reacting the strips with a chromogenic substrate for the enzyme.

gp160 can be purified from the mixture of glycoproteins eluted from lentil-lectin Sepharose® column by immunoaffinity chromatography using a mAb to HIV-1 gp41 protein. The mAb is produced and used employing procedures routine in the art. The gp160 elutes from the column in a nearly homogenous state as described in U.S. Pat. No. 5,116,740.

Glycoprotein gp160 and its derivatives, prepared as described are used to prepare vaccine compositions in accordance with the methods disclosed herein.

Trypanosomal Vaccine Peptides

The amino acid sequences of some peptides used to produce a suitable vaccine composition for this invention are given in TABLE 1, wherein trypanosomal peptides are exemplified. PepG is an example of a cyclic peptide—it has two cysteines which have been joined in a disulfide bond to make a cyclic loop in the peptide. PepM1 is non-cyclic, is without an added Cys and contains the native epitope only once. PepCM1 has an added Cys at the amino terminus as does pepCM3 and pepCM5. PepCM3 has three replicates of the native M epitope and pepM5 and pepCM5 have five such replicates. PepL1 has an epitope of only seven amino acids as does its Cys-containing counterpart, CL1.

Proteosomes and their Preparation

Proteosomes, in their native hydrophobic state, have special lymphocyte activating properties which allow them to act as both a protein carrier and an adjuvant. Since they are not chemically modified, but retain their multimolecular hydrophobic and membranous structure in the present vaccine composition, their ability to potentiate the immunogenicity of the antigenic peptides to which they are complexed is due to these special properties which are retained by the methods set forth herein.

Proteosomes may be prepared from Group B type 2b meningococcal cells. Proteosome preparation consists of two stages. The first stage may be done by (1) isolation of meningococcal outer membrane vesicles by extraction from an aqueous suspension of whole meningococci (Zollinger et al., J. Clin. Invest., 63, page 836-848, 1979)) or (2) collection of a direct bacterial cell extract precipitate. The direct cell extracts are typically obtained by extraction of packed bacterial cells for one hour at room temperature with 100 grams of cells per liter of a solution containing 0.1 M sodium acetate pH 5.0, 0.5 M CaCl₂ and 3% Empigen BB. Ethanol is added to the mixture to a concentration of 20% v/v and the precipitate removed by centrifugation at 16,000×g for 10 minutes. Additional ethanol is added to the supernatant to a final concentration of 45% and the precipitate, constituting the direct cell extract, is collected by centrifugation.

The second stage of the proteosome preparation consists of separating the outer membrane proteins from other membrane components by dissolving either of the products from the first stage (the vesicles or the direct cell extract) at a concentration of approximately 2 mg protein/ml in a buffer (hereafter referred to as TEEN-1%) The proteins were then precipitated three times by addition of solid ammonium sulfate at 500 g/l of protein solution. The precipitates were collected by centrifugation at 30,000×g for 20 minutes and redissolved at about 2 mg protein/ml in TEEN-1%. The final precipitate was dissolved with the aid of a water bath sonicator at about 2 mg/ml, centrifuged at 16,000×g for 20 minutes to remove insoluble material and then dialyzed against TWEEN-0.1% to remove any residual ammonium sulfate. (The final concentration of Empigen BB can be 0.1% to 1.0%). Products are stored at −20° C. (or, for short periods at 4° C.).

The proteosomes prepared from bacteria other than those prepared from meningococci may also be prepared and used by the same methodology.

In certain instances, the hydrophobic foot attached through the Cys may be sufficient to provide needed antigenicity without use of proteosomes.

Having now generally described the invention, the same will be more readily understood through reference to the following examples which are provided by way of illustration, and are not intended to be limiting of the present invention, unless specified.

### EXAMPLE I

### Responses to Vaccines Made from Trypanosomal Peptides

The results of several tests of the production and use of the present vaccine composition are detailed in TABLES 2-4. All vaccines were prepared as described below. Briefly, the peptides, with or without added cysteines, were synthesized by standard solid phase technology. While still on the resin, a lauroyl group was added to the amino terminus as described below or the pentapeptide hydrophobic foot, Phe-Leu-Leu-Ala-Val (FLLAV), was added by simply continuing the synthesis. Except when noted otherwise, all vaccines were prepared by dissolving the peptides and/or the proteosomes in TEEN-1% detergent buffer and then exhaustively dialyzing away the detergent.

As shown in TABLE 2, responses in two conventional strains of mice, BALB/c and C3H/HeJ, were compared. Among other differences, an important distinction between these strains is that C3H/HeJ mice are genetically non-responsive to lipopolysaccharide (LPS), an imrnunopotentiating substance and important component of many adjuvants. Immunization with pepG alone, pepG in Freund's adjuvant, pepG with proteosomes (but without any hydrophobic foot), or either lauroyl-pepg or FLLAV-pepG without proteosomes indicated a lack of immunogenicity (group 1, controls a-e). In marked contrast, pepG was made highly immunogenic by complexing with proteosomes via either a lauroyl hydrophobic foot (groups 2 and 4) or via the FLLAV pentapeptide hydrophobic foot (groups 3 and 5). This was demonstrated in both BALB/c mice (groups 2 and 3) and C3H/HeJ mice (groups 4 and 5).

Similarly, pepCL1 which is non-cyclic, was made immunogenic in both normal mice (group 15) and LPS non-responder mice (group 16). As expected, pepCL1 controls were non-immunogenic (group 13, controls a-d).

The M1 epitope was tested for immunogenicity in the system both with an added Cys (groups 9-12) and without the Cys (groups 6-8). Here, the Cys was found to be exceedingly important. High immunogenicity with either the standard 40 μg dose (group 11) or a sub-standard (8 μg) dose (group 12) of pepCM1 complexed to proteosomes. This peptide, lauroyl-pepCM1, was mildly immunogenic (after three immunizations) without proteosomes (group 10). In contrast, pepM1, lacking the Cys, exhibited only minimal immunogenicity even with proteosomes (groups 7 and 8). The Cys was considered to be important because its free sulfhydryl group causes dimerization of both the antigenic epitope and the hydrophobic foot. Dimerization of the epitope may allow better recognition by antigen presenting cells whereas dimerization of the hydrophobic foot promotes better complexing to proteosomes.

### EXAMPLE II

### The Role of Repeating Epitopes

The role of replicated epitopes in promoting immunogenicity is detailed in TABLE 3. Once again, the peptides alone, even when lengthened by repeating the epitope three times, were non-immunogenic (group 17, control groups a-c). Nevertheless, immunization of normal BALB/c mice with the fatty acyl hydrophobic foot variant of this peptide, lauroyl-pepCM3, indicated high immunogenicity even without proteosomes when the vaccine was prepared by the standard dialysis method (group 18). Note that lauroyl-pepCM3 was non-immunogenic when not dialyzed (group 19). Dissolving the lipopeptide in detergent and then dialyzing away the detergent is effective for promoting formation of auto-micelles during dialysis.

### EXAMPLE III

### Enhanced Immunogenicity Due to Proteosomes

When lauroyl-pepCM3 was complexed to proteosomes, immunogenicity was further enhanced (group 20). This vaccine was also effective in C3H/HeJ mice, demonstrating that the enhanced immunogenicity was not due to the <1% contaminating LPS in the proteosome preparation (group 24). It is believed that the low immunogenicity of lauroyl-pepCM3 in C3H/HeJ mice (group 23) was due to a genetically based lack of recognition of the M1 epitope in this strain and not due to the insensitivity to the adjuvant effect of LPS. This conclusion is based on the fact that (a) lauroyl-pepCM3 does not contain LPS, and (b) complexing this peptide to proteosomes, which are able to provide a carrier-like effect on T-cells, resulted in an immunogenic vaccine (group 24).

The optimal nature of the system when each of the four components (the proteosomes, hydrophobic foot, the Cys and the repeated epitopes) were present was also demonstrated using pepCM3 with the FLLAV pentapeptide hydrophobic foot. FLLAV-pepCM3 was not immunogenic alone (group 21) whereas FLLAV-pepCM3 complexed to proteosomes was among the most immunogenic of all vaccines using the M1 epitope (group 22).

The role of the Cys was also confirmed when it was tested in conjunction with repeated epitopes and the lauroyl hydrophobic foot. The pepM5 (group 25) and pepCM5 (group 28) controls were not immunogenic. When pepM5 was supplemented with the lauroyl hydrophobic foot (group 26) or both the lauroyl foot and proteosomes (group 27) mild immunogenicity was observed even though the peptide was 47 amino acids long and had 5 repeats of the M epitope. In marked contrast, the lauroyl-pepCM5 (which contains the added Cys) was highly immunogenic (group 29). Complexing lauroyl-pepCM5 to proteosomes further enhanced its immunogenicity (group 31) to maximal levels. When pepCM3 was incubated without dialysis, immunogenicity was markedly reduced (group 30). In C3H/HeJ mice, lauroyl-pepCM5 was only minimally immunogenic (group 32) but proteosome-lauroyl-pepCM5 was clearly immunogenic (group 33). These results are consistent with the results in C3H/HeJ mice described above.

### EXAMPLE IV

### Complexing Method and Proteosome:Peptide Ratio

As shown in TABLE 4, effective proteosome-hydrophobic foot vaccines can also be made without using the dialysis method. Although the dialysis method appeared to be optimal (groups 36-40), excellent immunogenicity was also obtained by lyophilizattion of a saline or water mixture of the peptide (containing a hydrophobic foot, e.g. lauroyl-CM1) with proteosomes that have previously been removed from the Empigen detergent (group 35). Simply mixing the components together in saline, was not as effective as either lyophilization or dialysis although significant immunogenicity was attained this way (group 34). There may be applications in which the alternate methodologies described would be advantageous.

Also shown in TABLE 4 is the effect of varying the ratio of proteosome to peptide in the vaccine from 1:1 to 1:16 (groups 3640). As is clearly evident, each of the vaccines was highly immunogenic. The advantages of being able to use a vaccine with a higher ratio of peptide per unit of proteosome protein are (a) less protein is needed to generate an effective immune response, thereby diminishing possible side effects; and (b) if a maximum amount of protein is administered, the amount of peptide that can be given is correspondingly increased. This increase in the amount of peptide that can be given may be critical to the development of a successful vaccine when the peptide epitope is particularly refractory to potentiation of immunogenicity.

### EXAMPLE V

### Responses to Plasmodial Vaccines Made from Repeating Peptides

An example of the value of the techniques disclosed herein is illustrated by use of a recombinant protein (R32RL), a 384 base pair fragment encoding 32 tetrapeptide repeats

[(Asn-Ala-Asn-Pro)₁₅ (Asn-Val-Asp-Pro)]₂ [SEQ ID NO:5]

of the P. falciparum CS protein, rendered immunogenic by adding the hydrophobic foot, Cys-containing decapeptide anchor to its carboxy terminus to create “R32Ft.” R32Ft is immunogenic in vaccine testing when used alone and such immunogenicity is markedly enhanced when it is complexed to proteosomes via the added hydrophobic decapeptide anchor described above.

Construction of the Anchored Recombinant Protein R32Ft:

Ten micrograms of expression vector pASI (ATCC 39262, more fully described in U.S. Pat. No. 4,578,355, which is incorporated herein by reference) was digested with restriction endonuclease BamHI (25 units) in 200 μl medium buffer [comprising 50 mM Tris, 5 mM NaCl, 1 mM dithiothreitol (DTT), and 10 mM MgCl₂, having a pH of 7.5] for 1.5 hours at 37° C. One hundred nanograms of the BamHI-cut pAS1 was ligated with 20 ng of a synthetic linker having the following sequence:

| | |
|---|---|
| 5′-GATCCCGGGTGACTGACTGA -3′ | SEQ ID NO: 6 |
| 3′- GGCCCACTGACTGACTCTAG-5′ | SEQ ID NO: 7 |

The resulting plasmid, pT17, was identified with one linker inserted into the BamHI site of pAS1. This vector retains the BamHI site, introduces a unique SmaI site, and results in the insertion of TGA termination codons in all three reading frames downstream of the ATG initiation codon of the cII ribosome binding site.

Four micrograms of purified pUC8 clone 1, a pUC8 clone (Viera, et al., Gene 19:259 (1982)) containing the CS protein coding sequence as a 2337 base pair EcoRI fragment of gamma-mPF1 inserted into the EcoRI site of pUC8 (Dame et al., Science 225:593 (1984)) was digested with restriction endonuclease XhoII in 400 μl of medium buffer for 1.5 hours at 37° C. The resulting 192 base pair fragment, encoding 16 tetrapeptide repeats [(Asn-Ala-Asn-Pro)₁₅-(Asn-Val-Asp-Pro)]₂ of the P. falciparum CS protein, was isolated by electrophoresis on a 5% polyacrylamide gel (PAGE) and recovered by electroelution.

Expression vector pT17 (10 μg) was digested with restriction endonuclease BamHI (25 units) in 200 μl medium buffer (described above) for 1.5 hours at 37° C. The Xho II CS protein gene fragment (1230 g) was then ligated into this vector (100 ng) in 30 μg ligase buffer (comprising 50 mM Tris, 1 mM DTT, 10 mM MgCl₂, and 100 μM rATP, having pH of 7.5) with one unit of T4-DNA ligase for 16 hours at 4° C.

The ligation mixture was transformed into E. coli strain MM294CI+ (SmithKline French]. Ampicillin resistant colonies were obtained and screened for insertion of the Xho II gene fragment into pT17. A plasmid with the correct construction, pR16, was identified and transformed into E. coli strain MM294CI+.

Expression vector pR16 was digested with restriction endonuclease BamHI as described above and a second Xho II CS protein gene fragment ligated into the vector. The ligation mixture was transformed into E. coli strain MM294CI+, ampicillin resistant colonies thereof selected and a plasmid with the correct construction, pR32, containing 32 repeats of the CS tetrapeptide, identified and transformed into E. coli strain MM294CI+.

Expression vector pR32 (10 μg) was digested by restriction endonucleases SmaI and SalI in 200 μl medium buffer (described above) for 1.5 hours at 37° C.

The synthetic DNA hydrophobic decapeptide anchor sequence (1 μg) identified below was then added and ligated to the SmaI/SalI cut pR32 (100 ng) in 30 μl ligase buffer with one unit of T4-DNA ligase at 4° C. for 16 hours. The hydrophobic decapeptide coding sequence was

5′ GGT GGT TAC TGC TTC GTT GCT CTG CTG TTC TGA G
3′ CCA CCA ATG ACG AAG CAA CGA GAC GAC AAG ACT
CAGCT

The ligation mixture was transformed into E. coli strain MM94CI+. Ampicillin resistant colonies were obtained and screened for the insertion of the decapeptide into pR32. A plasmid with the correct construction, pR32Ft, was identified and transformed into E. coli strain AR58 (CI⁸⁵⁷) and tested for expression of the gene product.

Cells were grown in Luria-Bertani Broth (LB) at 32° C to an absorbance at 650 nm (A₆₅₀) of 0.6 and were then induced at 42° C. for 3 hours to turn on transcription of the P_{L} promoter of the expression plasmid and subsequent translation of the CS protein derivative. Cells were sampled in 1 ml aliquots, pelleted, resuspended in lysis buffer (comprising 10 mM TrisHC1, 25% (vol/vol) glycerol, 2% 2-mercaptoethanol, 2% sodium dodecyl sulfate (SDS), and 0.1% bromophenol blue, having a pH of 7.8) and incubated in a 105° C. heating block for 5 minutes. Proteins were separated by SDS-PAGE (12% acrylamide, 30:0.8 acrylamide:bisacrylamide ratio). Protein produced from E. coli was detected by Western Blot analysis as described below.

Purification of R32Ft

The R32Ft peptide was purified from the above expression system as disclosed below. All operations were performed on ice unless stated otherwise. Three 20-g E coli frozen pellets (SmithKline Laboratories) were combined and thawed by suspending in 240 ml of 50 mM Tris, 2 mM EDTA, 5% glycerol [at pH 8.0 and stirring for one hour. Grade I lysozyme (48 mg, final concentration 0.2 mg/ml) and 1 ml phenylmethyl sulfonyl fluoride (PMSF) at a 34 mg/ml in absolute ethanol were added and the suspension stirred for 30 minutes. The lysate was blended for 1 one-minute intervals in a blender and sonicated for 3 one-minute intervals (Artek, model 300, medium probe). Sodium deoxycholate (DOC) was added to a final concentration of 0.1% (w/v). The suspension was stirred for 30 minutes, then centrifuged for 1 hour at 12000×g.

The supernatant was heated in a boiling water bath for 5 minutes with stirring, cooled for one hour at ambient temperature, and then centrifuged at 12000×g. Crude antigen was precipitated in a 10% to 40% ammonium sulfate pellet. The pellet was resuspended in 25 ml phosphate buffered saline (PBS) and dialyzed extensively against PBS (SpectroPor tubing, MW cut-off: 3000).

The sample was acidified to pH 2.0 by dropwise addition of 10% trifluoroacetic acid (TFA), stirred for 1 hour and centrifuged for 30 minutes at 12000×g. The supernatant was collected and dialyzed into 10% PBS and lyophilized to reduce the volume to 5 ml. The solution was recentrifuged to clarify it.

Final purification was carried out by high performance liquid chromatograph (HPLC) using a Waters system, including tow model 510 pumps, and model 481 detector, automated gradient controller and an LKB model 2212 Helirac fraction collector with a semi-prep C-3 reverse phase column. Protein elution was monitored at 214 nM. Buffer A was 0.05% TFA/water and Buffer B was 0.05% TFA in 90% MeCN/water. Flow was 9.5 ml/min. The gradient started at 70% A, proceeded linearly to 50% A in 20 minutes and was washed with 70% B for 8 minutes.

Proteins were neutralized by collection into equal volumes of saturated ammonium bicarbonate and assayed using a quick ELISA system. Protein peaks with strong ELISA activity were lyophilized and characterized by Western blot and amino acid analysis. Two peaks with activity were eluted consistently at 45% and 48% B. The proteins were indistinguishable by amino acid analysis and Western blot. Both exhibited a single band migrating at 54 kDa and the results of amino acid analysis were identical.

The anchored recombinant proteins were complexed to the proteosomes via dialysis. Proteosomes in a concentration of 0.5-2.5 mg/ml were added to solution of the recombinant protein with the hydrophobic foot to provide ratios of proteosomes to anchored recombinant protein (w/w) range of 1:1 to 1:20. The material was dialyzed in accord with the teachings above.

Animal Testing:

Previous attempts to immunize mice with the protein R32LR (without the hydrophobic sequence) showed this protein to be non-immunogenic, and it was only poorly immunogenic when administered with complete Freund's adjuvant or alum.

Groups of mice were dosed with (a) 50 μg proteosomes with 50-100 μg R32Ft or (b) 50-100 g R32Ft without proteosomes. All injections were given using saline as the carrier. No additional adjuvants were used. Analysis of pooled sera from the groups of mice showed that, while the recombinant R32Ft alone was effective, the recombinant R32Ft complexed with the proteosome was at least 16 fold more effective as a vaccine. Both C57BL/6 strain and BALB/c mice responded to the vaccines. When the animals were given booster injections (up to two boosters) an improved immune response was seen in all instances.

Individual rabbits were dosed with 100-200 μg R32Ft (recombinant) alone or complexed to 100 μg/dose of proteosomes. The recombinant R32Ft complexed with the proteosomes was about 10 fold as effective as the R32Ft having hydrophobic foot but no complexed proteosome.

Proteosomes can also be complexed with the anchored recombinant protein by lyophilization in accord with the methods taught above.

### EXAMPLE VI

### Response to Leishmania Vaccine

Mice immunized and then infected with Leishmania major in a murine model of cutaneous leishmaniasis having a lauryl or lauryl-Cys conjugated to the amino terminus was assessed for cell mediated immune response. Vaccines will consist of lauryl or lauryl-Cys conjugated to a selected synthetic gp63 peptide 467-482 having the following sequence (SEQ ID NO:8):

Gly-Asn-Val-Gln-Ala-Ala-Lys-Asp-Gly-Gly-Asn-Thr-Ala-Ala-Gly-Arg

The peptide covalently conjugated to lauryl-Cys protected against severe Leishmania cutaneous lesions with an average of 81% reduction of lesions in 3 separate experiments. This occurred even when giving the lauryl-cysteinyl-peptide in saline without other adjuvants whereas the cysteinyl-peptide or the peptide without the added lauryl moiety was ineffective. Addition of proteosomes or other peptides did not further enhance protection. Studying of proliferation were negative. GeneBank analysis of this peptide revealed a striking homology with a human integrin molecule responsible for localization of cellular elements in the inflammatory process, indicating that the parasite may use immune mimicry to avoid host defenses. This peptide may therefore have wide application in ameliorating pathologic cellular immune responses caused by other forms of Leishmania or other parasites or bacteria such as mycobacteria where protective cell-medicated immunity is important.

### EXAMPLE VIl

### Response to Staphylococcal Enterotoxin B Vaccine

Proteosomes confer intranasal immunogenicity on formalinized toxoid of Staphylococcal Enterotoxin B (SEB) when formulated with proteosomes. In mice anti-SEB respiratory IgA and serum IgG were induced when the complexed compositions in saline were administered intranasally. The proteosome-toxoid vaccine also showed enhanced immunogenically when given parenterally. The proteosome-toxoid vaccine was made by the dialysis method as described. The toxoid and proteosomes were mixed in the presence of 1% buffered detergent (Empigen) and dialyzed.

Mice immunized intranasally with proteosome-toxoid vaccines were significantly protected (p<0.0117) against systemic challenge with >4 LD₁₀₀ of SEB using the D-galactosamine SEB challenge model. Mice immunized parenterally with proteosome-toxoid vaccines responded with high levels of anti-SEB serum IgG which were further enhanced by adjuvant in with alum. Using the D-galactosamine model, 98% of the 55 mice immunized parenterally with these vaccines that induced high anti-SEB serum IgG were protected against parenteral SEB challenge whereas mice immunized with the formalinized toxoid in saline or alum that had titers <55,000 were significantly less protected.

As indicated, the methods disclosed herein are appropriate for use both with addition of the hydrophobic foot. However, when there is a hydrophobic moiety in or associated with the peptide, it is not necessary to synthetically add the hydrophobic foot.

### EXAMPLE VIII

### Response to HIV gp160-Proteosome Complex Vaccine in Rabbits

Proteosomes were constructed as indicated above and were stored at −70° C. in small aliquots at concentration of >5 mg/ml (usually 6-7 mg/ml) in TEEN-0. 1% buffer (or, on occasion, TEEN-1% having 1% Empigen BB). The proteosomes were defrosted immediately before use.

Prior to use of gp160 (in 0.01%TWEEN detergent), the antigenic material was prepared by one of the two following methods:

(1) Dialysis:

Seven ml of gp160 containing TWEEN was dialyzed across a SpectraPor membrane with molecular weight cutoff of 100 kDa against two liters of 0.1 M Tris buffered normal saline, pH 80 at 4° C. for four days, changing the buffer solution once per day. As an example, in one instance, 10.7 mg of gp160 in 0.1M Tris buffered saline was used of a stock of 0.54 mg/ml concentration in a volume of 19.8 mls. Next, Empigen BB was added to yield a final concentration of 1%.

The proteosomes were added to provide a 1:1 ratio (weight:weight) so that 10.7 mg of 6.7 mg/ml stock in 1.6 ml was added to produce a final concentration of 0.485 mg/ml of gp160 and proteosomes. The resulting product was dialyzed across a 1000 Da cut-off SpectraPor 6 or 7 membrane for 10 days at 4° C. against Tris buffered saline changing the buffer daily.

(2) Centrifugal Dialysis:

Centriprep 30 tubes were used to simultaneously remove the TWEEN and concentrate the gp160 stock from 0.7 mg/ml to >4 mg/ml by diluting 15 ml of the 0.7 ml stock with 5 ml of Tris buffered saline to yield a concentration of 0.5 mg/ml. This was centrifuged at 2,000 g in a Beckman centrifuge for 15 minutes at 4° C. to give 10 ml of partially concentrated gp160. This was diluted to 20 mls and recentrifuged as above, yielding a 10 ml volume. The resulting concentrate was rediluted with Tris buffered saline to 30 mls and recentrifuged as above to give a final volume of 3.2 mls with a gp160 concentration of 4.25 mg/ml (analyzed spectrophotometrically at A₂₈₀) and with an estimated 99.999% TWEEN removal and 94% recovery of gp160. For example, 7.2 mg of gp160 in 0.1M Tris buffered saline was used of a stock of 4.2 mg/ml concentration in a volume of 1.7 mls. Next, Empigen BB was added to give a final concentration of 1%.

The proteosomes were added to provide a 1:1 ratio (weight:weight) so that 7.2 mg of 6.7 mg/ml stock in 1.1 ml was added to result in a final concentration of 2.5 mg/ml of gp160 and proteosomes. The resulting product was dialyzed across a 1000 Da cut-off SpectraPor 6 or 7 membrane for 10 days at 4° C. against Tris buffered saline changing the buffer daily.

The gp160 is a much larger than the R32ft discussed above. The gp160 is also a transmembrane protein which naturally forms trimers that make its effective molecular weight even larger. The antigenic properties of compositions containing gp160 complexed to proteosomes can be enhanced by addition of adjuvants such as alum. It has also been discovered that submicron emulsions enhance immunogenicity.

TABLE 6 shows a comparison of ELISA analysis of sera from rabbits immunized 4 times i.m. with 85 μg of gp160 formulated with alum, proteosomes plus alum, or proteosomes plus sub-micron emulsions. The results indicate that the proteosome complex formulation resulted in a much higher titer response to an important gp120 epitope termed Alex 10.

### EXAMPLE

### Induction of IgG and IgA Mucosal and Serum Antibodies Using an Oligomeric gp160 Vaccine

Induction of IgG and IgA antibodies in vaginal, intestinal, and lung lavage fluids, fecal extracts and sera occurs following intranasal immunization of mice with an oligomeric gp160 vaccine. The vaccine can be delivered in saline, as a proteosome-oligo-gp160 complex alone or in a solid fat nanoemulsion. (See, for example, patent application U.S. Ser. No. 08/553,350 filed Nov. 16, 1995, for a method of preparing solid fat nanoemulsions; the content of this application are expressly incorporated herein by reference).

Proteosome preparation for oligo-gp160

Outer membrane protein proteosome preparations were purified as follows: Proteosomes were prepared from Group B type 2 Neisseria meningitides in two stages. The first stage was done by the collection of a bacterial cell extract precipitate. The direct cell extracts were obtained by extraction of packed bacterial cells by adding with one liter per 1000 grams of cell paste of a solution containing 1.0 M sodium acetate pH 5.0 mixing and then adding an equal volume of a solution of 1.0 M CaCl₂ with 6% Empigen BB. This suspension was then stirred for one hour at room temperature. Ethanol was added to the mixture to a concentration of 20% v/v, the precipitate removed by centrifugation at 10,800×g for 15 minutes and the supernatant was filtered through cotton gauze. Ethanol was added to the filtrate to a final concentration of 45% and the precipitate, constituting the direct cell extract, was collected by centrifugation at 10,800×g for 15 minutes.

The second stage of the proteosome preparation consisted of isolating the outer membrane proteins from the other membrane components by dissolving the product from the first stage (using a Teflon paste homogenizer followed by stirring and sonication) in a buffer (TEEN-1%) containing 0.05 molar Tris-HCl (hydroxyacetyl amino methane), 0.15 M NaCl, 0.01 M EDTA (ethylene diamine tetra-acetate) and 1% Empigen BB (Albricht and Wilson, Cubria, England) brought to pH 8.0 using 1.5 ml TEEN-1% per gram of initial paste. The suspension was then centrifuged at 13,800×g for 25 minutes at 4° C. The supernatant was saved and the dissolving process was repeated as above on the resultant pellet and subsequent pellets 24 times as needed, using 15-50% less volume as needed and saving and pooling all the supernatants which were then stored at 4° C. The proteins were then precipitated by the addition of solid ammonium sulfate at 500 g/l of protein solution. The pellet and solid precipitates were collected after centrifugation at 20-30,000×g for 20 minutes and redissolved in TEEN-1% using about 2 mg protein/ml or ¼-⅕ the volume of the first Teen-1% extraction. This procedure was repeated on the pellets twice more using 600 g/l of ammonium sulfate. The pellet from the last ammonium sulfate was dissolved using TEEN-1% at 1/4 the volume of the first Teen-1% extraction or 2 mg/ml with stirring and sonication. After centrifuging this solution at 28,400×g for 25 minutes, the supernatant was saved at 4° C. and the pellets were re-dissolved using less volume consonant with the size of the pellet. This process was repeated as needed until the pellet was negligible. To both remove residual ammonium sulfate, and concentrate the resultant proteosome proteins, the pooled dissolved pellets were dialyzed against TEEN-1% using an AG hollow fiber cartridge system with a 10,000 kDa cutoff A/G membrane using successive concentration and dilution 3-5 times. (The final concentration of Empigen BB can be 0.1% to 1.0%). Products are stored at −20° or −70° C. (or, for shorter periods at 4° C).

The final product can be filtered through a 0.22 micron membrane and the concentration of the resultant proteosome preparations can be from 1-10 mg/ml (4-7 mg/ml is preferred).

HIV (strain 451) Oligo-gp160 as previously described in U.S. Pat. No. 5,116,740.

Bioadhesive Nanoemulsions (pmax).

Mucoadhesive emulsion particles were prepared as previously described (see PCT/US/05589), 1:1 (w/w) fat/phospholipid mixture was dissolved in chloroform. The organic solvent was evaporated to complete dryness under reduced pressure using a rotary evaporator (Heidolph, Germany). To the dry lipid film, an aqueous solution containing 0.1% EDTA was added and the mixture-was then hydrated by shaking for 30 min. Using a multiwrist shaker (Labline, USA) until all the lipids were homogeneously dispersed in the aqueous phase. The dispersion was homogenized using a Microlab 70 Gaulin Homogenizer (5 cycles at 800 bar). Using an N4MT Particle size Analyzer (Coulter Electronics, England), the resultant emulsome particles were determined to have a mean diameter of 140+/−50 nm. Carbocol 934 (BF Goodrich, Atlanta, Ga.) was added (0.1% final concentration) to confer mucoadhesive properties to the emulsion preparation. Pmax were vigorously mixed with equal parts of rgp160 or proteosome-rgp160 preparations to result in a 2.5% lipid concentration in the vaccines containing the emulsion.

Preparation of Proteosome-oligo gp160 vaccine.

A portion of the stock oligo-gp160 was concentrated using an Amicon filtration unit with a 30,000 MWCO filter as needed and then complexed to and formulated with proteosomes using dialysis. The oligo-gp160, dissolved in saline buffered solution such as Dulbecco's PBS pH 7.4 containing Empigen BB (1%) and was then mixed with proteosomes at a 1:1 (wt/wt) ratio in the saline buffered 1% Empigen solution. The mixture was exhaustively dialyzed across a dialysis membrane with a 10,0000 Molecular Weight cutoff (SpectraPor 7; Spectrum Medical Industries, Los Angeles, Calif.) against buffered saline for 16-21 days at 4° C. exchanging at least 500 parts buffer each day and the vaccine was stored at 4° C. prior to and during the immunizations.

Preparation of oligo-gp160 or Proteosome-oligo-gp160 Vaccines with bioadhesive nanoemulsion. The oligo-gp160 or the proteosome-oligo-gp160 vaccine was vigorously mixed with equal volumes of the bioadhesive solid fat nanoemulsion prior to immunization.

Intranasal Immunizations

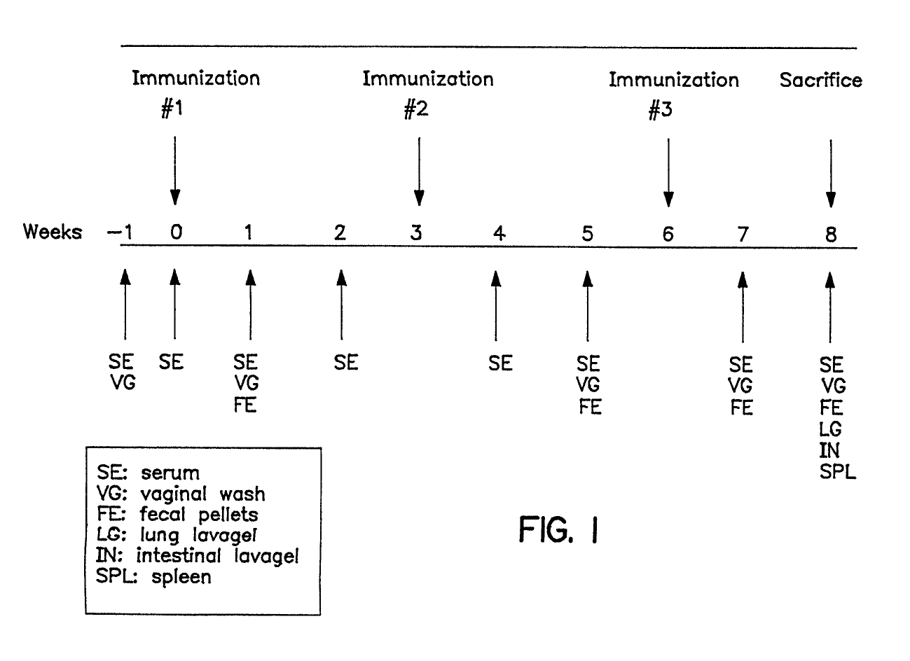

Female Balb/c mice were used throughout the experiment. Mice (five per group per experiment) were immunized intranasally and samples were collected according to the schedule shown in FIG. 1. Intranasal immunization was in volumes of 60 μl (divided into 30 μl applications spaced 2-4 hours apart) for each of the three immunizations (at 3 week intervals) with preparations containing 10 or 50 μg or oligo-gp160 formulated with either (1) saline, (2) bioadhesive nanoemulsion, (3) proteosomes in buffered saline, (4) proteosomes plus bioadhesive nanoemulsion. Prior to immunization, mice were mildly anaesthetized as previously described (Lowell et al. (1996) Infection and Immunity 64:1706-1713) or with a mixture of zylazine and ketamine or with methoxyflurane and then allowed to inhale 25-35 μl of the vaccine or saline (for non-immunized control animals) that was slowly instilled by micropipette into both nares.

Sample Collection

This was performed as shown in FIG. 1. To prepare serum, blood was collected 7-14 days (and also, at times several weeks) after each immunization and stored individually at −70° C. Assays were performed on pooled samples using equivalent volumes of freshly defrosted samples of sera from each of the five mice in the group.

Vaginal secretions. Samples were collected as previously described in detail using wicks (Polyfiltronic Group Inc., Rockland, Mass.) inserted prior to sacrifice, 10-14 days after the third immunization. The day of wick insertion was timed to coincide with the estimated time of ovulation which had been previously synchronized by piacing a male mouse in a nearby cage on the appropriate day. briefly, the wicks were inserted after instillation of 25 μL PBS intravaginally and allowed to absorb secretions for 30-60 seconds after which the wick was removed, an additional 25 μL PBS was instilled and the opposite end of the wick was inserted into the vagina for another 30-60 seconds. The wick was transferred to a microfuge tube, immediately frozen with dry ice and stored individually at 70° C. Secretions from each mouse were individually eluted from the wicks by adding 0.8 mL of a solution of 0.5% each of BSA and casein plus protease inhibitors to the tube with the wick. The tube was then centrifuged at 10,000 rpm for 15 minutes prior to sampling for the ELISA.

Intestinal and lung lavage fluids. Secretions were collected at sacrifice as previously described (Lowell et al. (1996) Infection and Immunity 64:1706-1713), 14 days after the third immunization. Briefly, for bronchial lavage samples, immediately after sacrifice by CO₂ suffocation, the lungs were surgically exposed, a cannula was inserted in the trachea and, using a three-way stopcock, two lung lavage samples (1 mL each) using PBS containing 0.1% BSA were collected and combined. Intestinal lavage samples were then collected as described (Lowell et al. (1996) Infection and Immunity 64:1706-1713) by passing 2 mL of PBS containing 0.1% BSA, 50 mM EDTA and 1 mg/mL of soybean trypsin inhibitor through a 20-25 cm section of small intestine. Lavage fluids from each mouse were vortexed and centrifuged to remove cell debris and then individually stored at −70° C.

Fecal extracts

Fecal pellets (25-30) were collected and pooled from groups of five mice one week after the last immunization. each collected pool of pellets was weighed and PBS containing 0.1% Sodium Azide was added to the pellets at a ratio of 1 mL per 0.1 g fecal pellets. The samples were vortexed vigorously for 15 minutes and then centrifuged in a microfuge at 14,000 RPM for 15 minutes to remove debris prior to storage of the supernatant at −20° C.

Antibody Detection. ELISA

Sera, vaginal fluids and fecal extracts (representing rectal or lower intestinal antibody secretion) and sera were collected prior to immunization and after each immunization as described above; intestinal and lung lavage fluids were collected after the last immunization as described above. An ELISA was performed using oligo-gp160 as the detecting antigen. Briefly, 96 well round bottom microtiter plates (Immulon 2, Dynatech, Chantilly, Va.) were coated with oligo-gp160 using a solution of oligo-gp160 at 8-10 μg/μL and incubated at 37° C. for 1 hour. All incubations were performed in a humid chamber. After aspiration of the antigen using a plate washer (Skatron, Inc., Sterling, Va.), plates were washed once with PBS containing 0.05% Tween (PBS-T) and incubated with blocking solution containing 0.5% each of casein and BSA (IgG and fatty acid-free) for 60-90 min. at 37° C. After aspirating the blocking solution and washing twice with PBS-T, duplicate samples of sera, intestinal lavage fluids, bronchial lavage fluids, vaginal secretions or fecal extracts, serially diluted 2-fold in blocking solution, were added and the plates were incubated overnight at 37° C. After washing four times with PBS-T, affinity purified labeled (e.g. with horseradish peroxidase (HRP)) goat anti-mouse IgG or IgA (Kierkegaard and Perry Laboratories, Inc., Gaithersburg, Md.) was added and plates were incubated at room temperature (r.t.) overnight. After aspirating and washing twice with PBS-T, developing solution e.g. TMB solution (BioRad) was added and plates were incubated (for the periods described below) at r.t. prior to determining the absorbance values at e.g. A₆₃₀ using a microtiter ELISA plate reader.

Absorbances in the ELISA plate reader were determined after 15-30 min. Antibody titers in these Figures are expressed as the greatest dilution of sera, lavage fluids or collections of vaginal secretions with O.D. values >0.2.

Anti-oligo-gp160 IgG and IgA antibodies in each of the fluids, sera and extracts collected from mice immunized with oligo-gp160 delivered with proteosomes and/or nanoemulsions were analyzed by ELISA and compared to the collections of samples from saline immunized animals and from animals immunized with oligo-gp160 delivered alone without proteosomes or the nanoemulsion. Results are shown in TABLE 6A and 6B.

Results

The results show that respiratory immunization with oligo-gp160 formulated with any of the following: (1) proteosomes in saline, (2) proteosomes in the bioadhesive nanoemulsion (pmax) or (3) pmax without proteosomes each enhance specific anti-oligo-gp160 IgG and IgA antibody formation in each of the samples compared to immunizing with the oligo-gp160 without either proteosomes or the bioadhesive nanoemulsion (see Table 6A-6B). Especially for induction of IgG and IgA antibodies in fecal pellets (which reflects rectal or lower intestinal antibody secretion) or in vaginal secretions, (or in lung or intestinal lavage fluids, as shown), proteosome formulation was preferred and the combination of proteosomes with the bioadhesive nanoemulsion was most preferred (Tables 1 and 2).

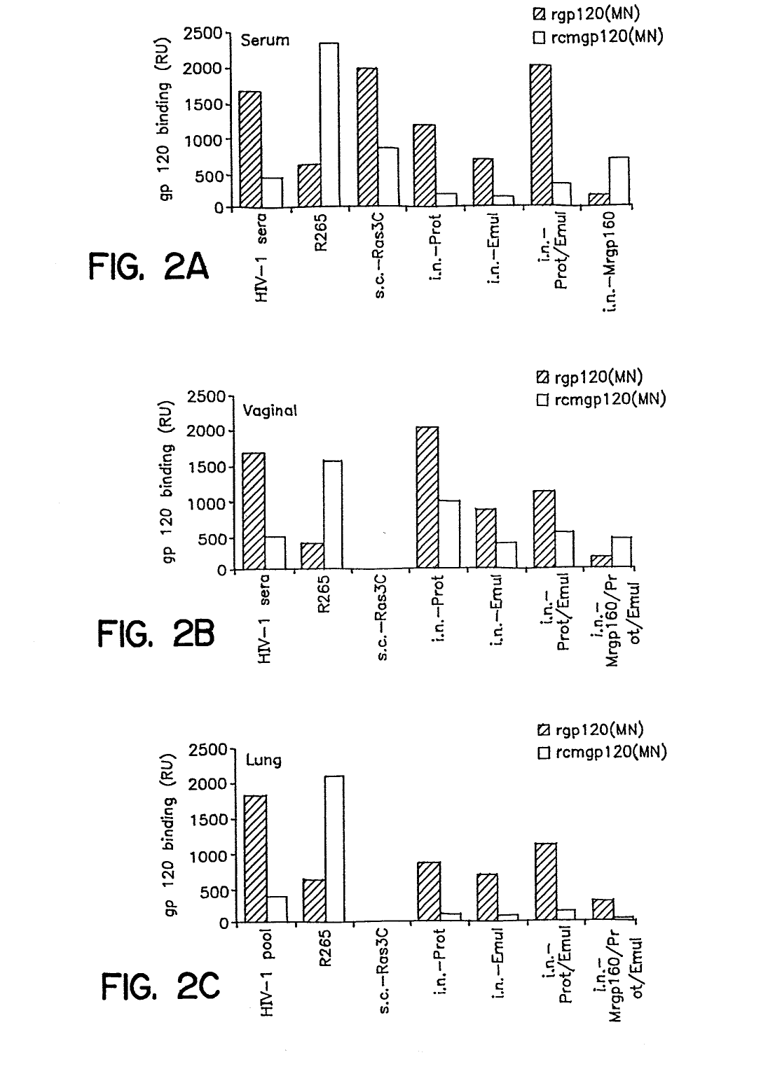

Respiratory immunization with oligo-gp160 formulated with proteosomes (prot), bioadhesive nanoemulsion (emul or pmax) (alone) or proteosomes plus bioadhesive nanoemulsion (prot/emul) induce antibodies in serum, vaginal wash and lung wash that preferentially reacted with the natively folded HW gp120 compared to their recognition of reduced and carboxymethylated gp120 (rcmgp120) (FIGS. 2A-2C).

Respiratory immunization with oligo-gp160 formulated with proteosomes (prot) or proteosomes plus bioadhesive nanoemulsion (prot/emul) clearly elicit local production of HIV-1 gp160 specific IgG and IgA responses (Table 8). This was demonstrated by dividing the HIV-1 gp160 specific endpoint titer in serum and mucosal washes and dividing by the measured total local IgG and IgA concentrations. These date indicate that antibodies within the mucosal washes were derived principally from local antibody production with minimal contribution from serum transudate.

Induction of neutralizing antibodies in vaginal and lung lavage fluids as well as in sera following intranasal immunization:

Mice were immunized and fluids and sera were collected as in Induction of IgG and IgA antibodies section, above and detailed in FIG. 1.

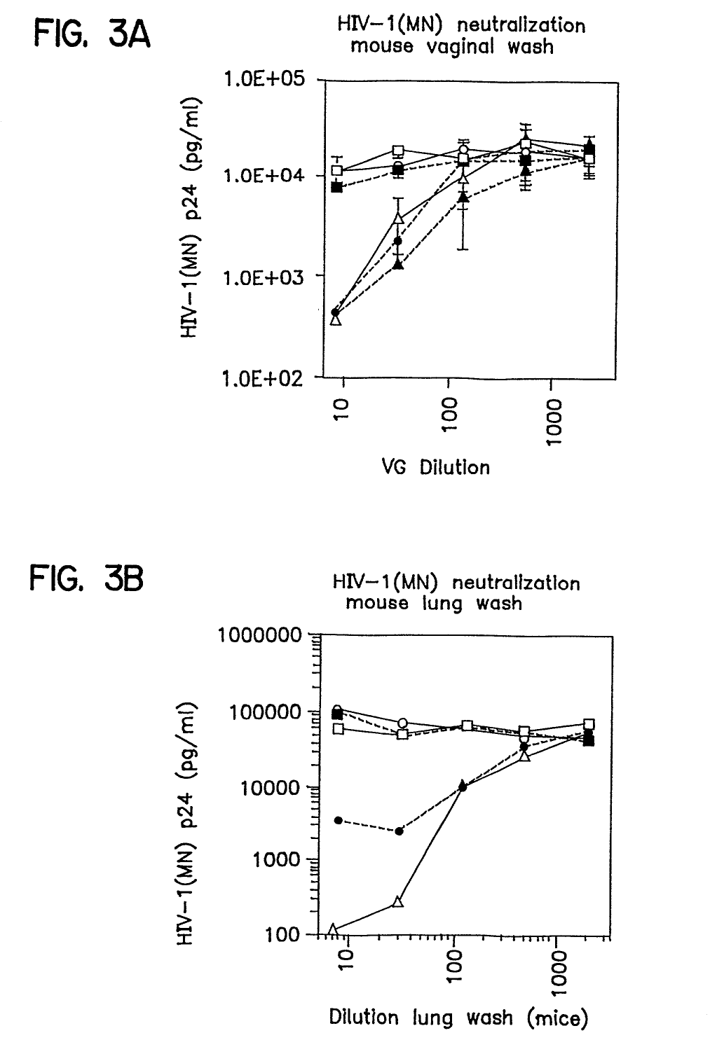

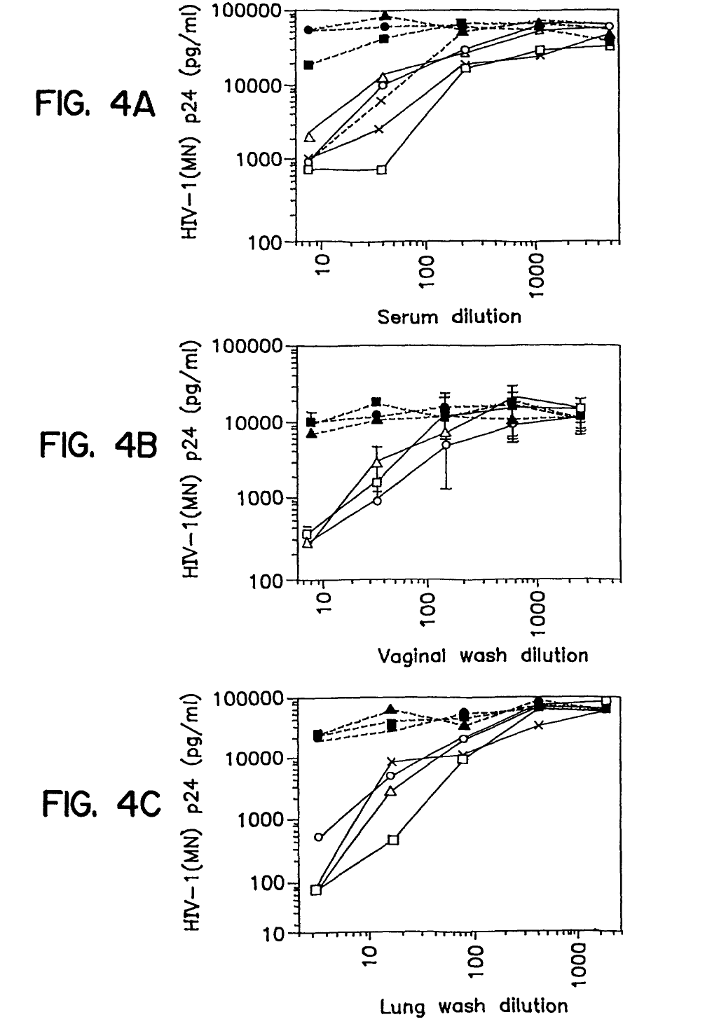

Induction of neutralizing antibodies in vaginal and lung lavage fluids as well as in sera following intranasal immunization of mice with the proteosome-oligo-gp160 vaccine in saline or the proteosome oligo-gp160 vaccine delivered in a bioadhesive solid fat nanoemulsion (pmax) (FIGS. 3A, 3B, FIGS. 4A-4C). As shown by the 1.5-3 log reductions in viral titers in vitro (as measured by the p24 assay (pg/ml)), neutralizing antibody in vaginal fluid (VG) (FIG. 3A), lung fluid (LG) (FIG. 3B) and in sera (FIGS. 4 and 5) were induced by the proteosome-oligo-gp160 vaccine delivered in saline or the proteosome-oligo-gp160 vaccine formulated with the bioadhesive nanoemulsion (pmax). Vaginal (VG) or lung (LG) fluids from saline controls or pre-immunization sera were unable to elicit antibodies that neutralized the virus as shown by the lack of reduction in viral titers (in the p24 assay) using these control samples.

TABLE 1
Amino Acid Sequences of Several Trypanosomal
Peptides Tested in the Proteosome-Hydrophobic
Foot Vaccine System

| No. | CODE | SEQUENCE | SEQ ID NO:. |
|---|---|---|---|
| 1 | pepG | YGG (GCTQITEPTCN)—S-S— | 9 |
| 2 | pepM1 | YC (VPVATQTG) | 10 |
| 3 | pepCM1 | CYG (VPVAQTQTG) | 11 |
| 4 | pepCM3 | CYG (VPVAQTQTG)₃ | 12 |
| 5 | pepM5 | YG (VPVAQTQTG)₅ | 13 |
| 6 | pepCM5 | CYG (VPVAQTQTG)₅ | 14 |
| 7 | pepL1 | (KYNATKA) | 15 |
| 8 | pepCL1 | C (KYNATKA) | 16 |

The sequences within parentheses are identical to the sequences of the peptides in the native organism.

TABLE 2
Anti-Peptide Antibody Titers In Sera of Mice After Primary (1°),
Secondary (2°) and Tertiary (3°) Immunization with Peptides with
Lauroyl Or FLLAV Hydrophobic Feet and/or Cysteine and/or Proteosomes

| | | | SERUM ANTIBODY | | |
|---|---|---|---|---|---|
| | | | ANTI-PEPTIDE TITERS | | |
| | MOUSE | IMMUNIZATION | POST VACCINE | | |
| NO. | STRAIN§ | WITH: | 1° | 2° | 3° |
| 1 | B,J | pepG Controls (a-e)* | <50 | <50 | <50 |
| 2 | B | Proteosome-Lauroyl- | 400 | 204,800 | 204,800 |
| | | pepG | | | |
| 3 | B | Proteosome-FLLAV- | 400 | 12,800 | 102,400 |
| | | pepG | | | |
| 4 | J | Proteosome-Lauroyl- | 200 | 6,400 | 51,200 |
| | | pepG | | | |
| 5 | J | Proteosome-FLLAV- | 100 | 102,400 | 409,600 |
| | | pepG | | | |
| 6 | B | pepM1 Controls (a-c)* | <50 | <50 | <50 |
| 7 | B | Lauroyl-pepM1 | <50 | 200 | 400 |
| 8 | B | Proteosome-Lauroyl- | <50 | 400 | 400 |
| | | pepM1 | | | |
| 9 | B | pepCMl Controls | <50 | <50 | <50 |
| | | (a-c)* | | | |
| 10 | B | Lauroyl-pepCM1 | <50 | <50 | 3,200 |
| 11 | B | Proteosome-Lauroyl- | 400 | 102,400 | 409,600 |
| | | pepCM1 | | | |
| 12 | B | Proteosome-Lauroyl- | 200 | 102,400 | 204,900 |
| | | pepCM1 (8 ug) | | | |
| 13 | B,J | pepCL1 Controls | <50 | <50 | <50 |
| | | (a-d)* | | | |
| 14 | B | Lauroyl-pepCL1 | 800 | 400 | 800 |
| 15 | B | Proteosome-Lauroyl- | 50 | 200 | 51,200 |
| | | pepCL1 | | | |
| 16 | J | Proteosome-Lauroyl- | 50 | 400 | 51,200 |
| | | pepCL1 | | | |

§Groups of 5-8 BALB/c (B) or C3H/HeJ (J) mice were immunized ip on weeks 0, 3 and 7 with vaccines containing 40 μg of peptide; sera, obtained 2-3 weeks after each immunization, were tested in an ELISA for IgG antibodies against the homologous peptide (either pepG, pepM1 or pepL1). Titers are the highest serum dilutions which had ELISA values that were a) more than 0.1 OD units and b) twice the value of pre-vaccination sera diluted 1:50.
*Each of the Control groups consisted of 5 mice immunized with either a) peptide alone, b) peptide in Freund's adjuvant, c) peptide and Proteosomes without hydrophobic feet, d) Lauroyl peptide without proteosomes, and e) FLLAV-peptide without Proteosames.

TABLE 3
Anti-Peptide Antibody Titers in Sera of Mice After Primary (1°),
Secondary (2°) and Tertiary (3°) Immunizations with Peptides with
Lauroyl or FLLAV Hydrophobic Feet and/or Cysteines
and/or Replicated Epitopes and/or Proteosomes

| | | | ANTI-PEPTIDE SERUM | | |
|---|---|---|---|---|---|
| | | | ANTIBODY TITERS POST- | | |
| | MOUSE | | IMMUNIZATION | | |
| GRP | STRAIN | VACCINE | 1° | 2° | 3° |
| 17 | B, J | pepCM3 Control groups (a-c)* | <50 | <50 | <50 |
| 18 | B | Lauroyl-pepCM4 | 400 | 102,400 | 102,400 |
| 19 | B | Lauroyl-pepcM3 (non-dialyzed) | <50 | <50 | 100 |
| 20 | B | Proteosome-Lauroyl-pepCM3 | 6,400 | 102,800 | 409,600 |
| 21 | B | FLLAV-pepCM3 | <50 | 50 | 50 |
| 22 | B | Proteosome-FLLAV-pepCM3 | <50 | 204,800 | 6,553,600 |
| 23 | J | Lauroyl-pepCM3 | <50 | 50 | 50 |
| 24 | J | Proteosome-auroyl-pepCM3 | <50 | 800 | 204,800 |
| 25 | B | pepM5 Control groups (a-c)* | <50 | <50 | <50 |
| 26 | B | Lauroyl-pepM5 | 200 | 400 | 12,800 |
| 27 | B | Proteosome-Lauroyl-pepM5 | 200 | 1600 | 12,800 |
| 28 | B, J | pepCM5 Control groups (a-c)* | <50 | <50 | <50 |
| 29 | B | Lauroyl-pepCM5 | 800 | 204,800 | 204,800 |
| 30 | B | Lauroyl-pepCM5 (non-dialyzed) | 100 | 12,800 | 25,600 |
| 31 | B | Proteosome-Lauroyl-pepCM5 | 400 | 15,600 | 3,276,800 |
| 32 | J | Lauroyl-pepCM5 | 50 | 100 | 100 |
| 33 | J | Proteosome-Lauroyl-pepCM5 | 200 | 25,600 | 51,200 |

§ Groups of 5-8 BALB/c (B) or C3H/HeJ (J) mice were immunized ip with vaccines containing 40 μg of peptide on weeks 0, 3 & 7; sera, obtained 2-3 weeks after each immunization, were tested in an ELISA for anti-pepMI IgG. Titers shown are the highest serum dilutions with ELISA values that were both a) > 0.1 O.D. units and b) twice the value of pre-vaccination sera diluted 1:50.
*Each of the Control groups consisted of 5 mice immunized with either (a) peptide alone, (b) peptide in Freund's adjuvant, (c) peptide and proteosomes without hydrophobic feet, (d) lauroyl peptide without proteosomes, and (e) FLLAV-peptide without proteosomes.
*The detergent (Empigen) was removed from the proteosomes by ethanol precipitation and the proteosomes were washed and resuspended in saline prior to mixing (group 34) or lyophilization (group 35) with a saline solution of pepCM1.
§ Groups of 5-8 C57B1/6 mice were immunized ip on weeks 0, 3 and 7 with 40 μg of peptide and the corresponding amount of proteosomes obtained 2-3 weeks after each immunization, were tested in an ELISA for IgG antibodies directed against the homologous peptide, pepM1; titers shown are the highest serum dilutions that had ELISA values that were both a) greater than 0.1 O.D. units and b) twice the value of pre-vaccination sera diluted 1:50.

TABLE 4
Effects of the Complexing Method and the
Proteosome:Peptide Ratio on the Ability of
Proteosomes to Enhance the Immunogenicity of Peptide Lauroyl-Cm1

| | | | ANTI-PEPTIDE SERUM | | |
|---|---|---|---|---|---|
| | | | ANTIBODY TITERS POST | | |
| GRP | Method of | Proteosome: | IMMUNIZATION§ | | |
| No. | Complexing | Peptide RATIO | 1° | 2° | 3° |
| 35 | Lyophilize | 1:1 | 800 | 12,800 | 409,600 |
| 34 | Mix | 1:1 | 400 | 6,400 | 51,200 |
| 36 | Dialyze | 1:1 | 12,800 | 409,600 | 6,553,600 |
| 37 | Dialyze | 1:2 | 25,600 | 819,200 | 819,000 |
| 38 | Dialyze | 1:4 | 6,400 | 819,200 | 1,638,400 |
| 39 | Dialyze | 1:8 | 12,800 | 819,200 | 1,638,400 |
| 40 | Dialyze | 1:16 | 51,200 | 1,638,400 | 3,276,800 |

§ Groups of 5-8 BALB/c or C3H/HeJ mice were immunized ip on weeks 0, 3 and 7 with vaccines containing 40 μg of peptide; sera, obtained 2-3 weeks after each immunization, were tested in an ELISA for IgG antibodies against meningococcal outer membrane proteins. Titers shown are the highest serum dilutions obtained after two or three immunizations which had ELISA values that were (a) more than 0.1 OD. units and (b) twice the value of pre-vaccination sera diluted 1:50.

TABLE 5
Anti-Meningococcal IgG Antibodies in Sera of Mice Immunized
and Boosted with Proteosome-Hydrophobic Foot-Peptide
Vaccines Using Either Lauroyl or FLLAV Hydrophobic Foot

| GRP | | Anti-Meningococcal IgG |
|---|---|---|
| NO. | Immunization | Vaccine Antibody Titers § |
| 1 | Controls | <50 |
| 2 | Proteosome-Lauroyl-pepG | 102,400 |
| 4 | Proteosome-FLLAV-pepG | 409,600 |

§ Groups of 5-8 BALB/c or C3H/HeJ mice were immunized ip on weeks 0, 3 and 7 with vaccines containing 40 μg of peptide; sera, obtained 2-3 weeks after each immunization, were tested in an ELISA for IgG antibodies against meningococcal outer membrane proteins. Titers shown are the highest serum dilutions obtained after two or three immunizations which had ELISA values that were (a) more than 0.1 OD unit and (b) twice the value of pre-vaccination sera diluted 1:50.

TABLE 6
ENHANCED SERUM ANTIBODY RESPONSE TO THE GP160
ANTIGENS INDUCED IN RABBITS BY FORMULATING
GP160 WITH PROTEOSOMES

| | Geometric mean of serum IgG titers | | |
|---|---|---|---|
| Vaccine preparation | gp160 | gp41 | Alex 10* |
| gp160/alum | 30,274 | 680 | 1 |
| gp160/protesosome/alum | 51,112 | 565 | 693 |

*Alex 10 is a significant epitope of gp120.

TABLE 7
ELISA Titers of Sera from Mice Immunized with oligo-gp 160 formulated with:

| | | | | | | | | | |
|---|---|---|---|---|---|---|---|---|---|
| | | Saline | | proteos/ | proteos/ | saline | | proteos/ | proteos/ |
| | No. of | control | emulsion | saline | emulsion | control | emulsion | saline | emulsion |
| Sample | doses | IgG | IgG | IgG | IgG | IgA | IgA | IgA | IgA |

A. 50 μg oligo gp 160

| | | | | | | | | | |
|---|---|---|---|---|---|---|---|---|---|
| serum | 2 | 256,000 | 16,000,000 | 16,000,000 | >8,000,000 | 400 | 3,200 | 12,800 | 12,800 |
| | 3 | 819,000 | 3,200,000 | 6,500,000 | 3,200,000 | 3,200 | 25,600 | 25,600 | 51,200 |
| vaginal secretions | 2 | 4 | 512 | 4,096 | 16,000 | <2 | 1,024 | 4,096 | 16,000 |
| | 3 | <2 | 1,024 | 16,000 | 128,000 | <2 | 2,048 | 16,000 | 64,000 |
| fecal pellets | 2 | 16 | 32 | 64 | 256 | 4 | 16 | 32 | 32 |
| | 3 | 4 | 512 | 4,096 | 8,192 | 16 | 256 | 512 | 2,048 |
| intestinal lavage | 3 | 16 | 8,192 | 32,000 | 64,000 | 16 | 256 | 512 | 1,024 |
| lung lavage | 3 | 512 | 8,192 | 16,000 | 32,000 | 2 | 256 | 512 | 256 |

B. 10 μg oligo-gp 160

| | | | | | | | | | |
|---|---|---|---|---|---|---|---|---|---|
| serum | 2 | 51,000 | 4,000,000 | 8,000,000 | 16,000,000 | 100 | 12,800 | 3,200 | 12,800 |
| | 3 | 51,000 | 3,200,000 | 4,00,000 | 13,100,000 | 800 | 12,800 | 6,400 | 800 |
| vaginal secretions | 2 | 128 | 256 | 1,024 | 16,000 | 32 | 256 | 2,048 | 16,000 |
| | 3 | 32 | 1,000 | 4,026 | 128,000 | 32 | 2,048 | 2,048 | 64,000 |
| fecal pellets | 2 | 64 | 16 | 16 | 256 | <2 | 8 | 16 | 32 |
| | 3 | <2 | 128 | 512 | 8,192 | 32 | 64 | 256 | 2,048 |
| intestinal lavage | 3 | 8 | 8,192 | 16,000 | 16,000 | <2 | 256 | 128 | 512 |
| lung lavage | 3 | 4 | 16,000 | 8,192 | 8,192 | <2 | 128 | 128 | 256 |

TABLE 8
IgG and IgA Antibody Specific Activity for oligo-gp160(451)in Serum and Mucosal Washes of Mice
Immunized Subcutaneously or Intranasally with Oligomeric gp160^{a}

| Group | Serum | | Vaginal | | Lung | | Intestinal | | Fecal | |
|---|---|---|---|---|---|---|---|---|---|---|
| # | IgG | IgA | IgG | IgA | IgG | IgA | IgG | IgA | IgG | IgA |
| Subcutaneous | | | | | | | | | | |
| Ras3C | 359 | 4 | <0.7 | <0.7 | <1.5 | — | <0.6 | <0.03 | <1.6 | <0.01 |
| Ras3C | 4630 | 3 | <1.0 | <1.2 | — | — | <2.6 | <0.02 | <7.1 | <0.01 |
| Ras3C | 5920 | 13 | <6.7 | <4.3 | — | — | <2.6 | <0.02 | <7.1 | <0.01 |
| Intranasal | | | | | | | | | | |
| saline-50 | 760 | 5 | 4 | <0.7 | 41 | 57 | 3,560 | 0.1 | 751 | 1.9 |
| saline-15 | 41 | 1 | 77 | 24^{b} | — | — | 36 | <0.02 | 76 | 0.1 |
| prot-50 | 1,800 | 24 | 30,300 | 9,510 | 12,800 | 552 | 5,070* | 195 | 25,100 | 2.4 |
| prot-15 | 3,610 | 8 | 7,470* | 2,650 | 13,400* | 216 | 138,000 | 3.7 | 3,750 | 1.4 |
| prot/emul-50 | 2,400 | 22 | 453,000 | 61,000 | 74,600 | 460 | 89,800 | 147 | 69,600 | 13.7 |
| prot/emul-15 | 1,620 | 4 | 350,000 | 22,700 | 7,910 | 315 | 12,000 | 74 | 17,400 | 3.2 |

^{a}Specific activities were calculated by dividing the specific anti-o-gp160 endpoint titer by the total Ig concentration (μg/ml) in each wash or serum. Dashed lines indicate no detectable Ig within the wash. VG refers to vaginal fluids. LG refers to lung fluids. IN refers to intestinal fluids. FE refers to fecal extracts.
The following mean total IgG and IgA concentrations were determined: serum IgG (2569 ± 1892); serum IgA (841 ± 399); VG IgG (0.48 ± 0.34); VG IgA (1.14 ± 0.52); LG IgG (0.53 ± 0.48) LG IgA (0.54 ± 0.69); IN (0.60 ± 1.31); IN IgA (33.9 ± 17.2); FE IgG (0.18 ± 0.13); FE IgA (158 ± 47).
^{b}Values shown in bold and italic represent an increase of at least 5-fold and values marked by an * represent an increase of between 2- and 5-fold, in specific activity compared to serum. Increases of this magnitude in local or regional IgG or IgA production serve as proof that the antibodies are produced locally and are not a result of serum transudation or blood contamination during preparation of the vaginal, intestinal, lung or fecal material.

All documents cited above are herein incorporated by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth as follows in the scope of the appended claims.

What is claimed is:

1. A vaccine composition capable of eliciting neutralizing antibodies in a subject to a pathogenic organism which antibodies are present in vaginal secretions, intestinal secretions, lung secretions or feces, which composition comprises:
(a) an antigen comprising a protein or peptide having
(i) an endogenous hydrophobic sequence of between about 3 and about 50 non-polar or uncharged amino acids;
(ii) added to the protein or peptide, an exogenous hydrophobic material comprising a sequence of between about 3 and about 50 non-polar or uncharged amino acids or a C8-C18 fatty acyl group; or
(iii) both (i) and (ii),
(b) complexed with said antigen, a composition comprising proteosomes, bioadhesive nanoemulsions, or both,
wherein said complexed or coupled protein or peptide maintains a native structure of antigenic epitopes such that, upon administration to said subject, the antigen induces neutralizing antibodies in one or more of vaginal secretions, intestinal secretions, lung secretions and feces, capable of neutralizing said pathogenic organism.

2. A vaccine composition according to claim 1 wherein the endogenous hydrophobic sequence or the exogenous hydrophobic material is a sequence of about 5 to about 29 amino acids.

3. A vaccine composition according to claim 1 wherein the exogenous hydrophobic material is a C8-C18 fatty acyl group.

4. A vaccine composition according to claim 3 wherein the exogenous hydrophobic material is lauroyl.

5. A vaccine composition according to claim 1 wherein the exogenous hydrophobic material is Phe Leu Leu Ala Val or Val-Ala-Leu-Leu-Phe.

6. A vaccine composition according to claim 1 wherein the antigen is a is a peptide or peptide oligomer.

7. A vaccine composition according to claim 1 wherein the protein is a viral envelope protein

8. A vaccine composition according to claim 5 wherein the viral envelope protein is an oligomeric gp160 from human immunodeficiency virus.

9. A vaccine composition according to claim 8 wherein said oligomeric gp160 has the sequence of residues 33-681 of SEQ ID NO:1.

10. A vaccine composition according to claim 1 wherein the protein or peptide is recombinantly produced.

11. A vaccine composition according to claim 1 wherein the antigenic protein or peptide natively contains at least one cysteine residue or has at least one added cysteine residue.

12. A vaccine composition according to claim 1 wherein the proteosomes are hydrophobic, multimolecular membrane proteins

13. A vaccine composition according to claim 1 formed by:
(a) bonding the hydrophobic material to said protein or peptide to form a hydrophobic-hydrophilic compound; and
(b) admixing said compound with said proteosomes, bioadhesive nanoemulsions, or both such that said antigen is complexed with said proteosomes or nanoemulsion.

14. A vaccine composition according to claim 13 wherein said admixing step is performed in the presence of a detergent, and is followed by the step of
(c) removing the detergent by dialysis.

15. A vaccine composition according to claim 13 wherein said admixing step is performed lyophilization.

16. A vaccine composition according to claim 1 formulated for intranasal or respiratory administration.

17. A vaccine composition according to claim 1 wherein the vaccine is in a dosage form suitable for multiple inoculations.

18. A vaccine composition according to claim 1 wherein the pathogenic organism is a causative agent of a mucosally-transmitted or sexually transmitted disease.

19. A process for inducing a neutralizing antibody response in a subject against a pathogenic organism resulting in neutralizing antibodies in one or more of vaginal secretions, intestinal secretions, lung secretions and feces, which process comprises administering to the subject an effective amount of a vaccine composition according to claim 1.

20. A process according to claim 19 wherein the exogenous hydrophobic material of said vaccine composition is a C8-C18 fatty acyl group.

21. A process according to claim 19 wherein the exogenous hydrophobic material of said vaccine composition is lauroyl, Phe Leu Leu Ala Val or Val-Ala-Leu-Leu-Phe.

22. A process according to claim 19 wherein the protein is a viral envelope protein.

23. A process according to claim 22 wherein the viral envelope protein is an oligomeric gp160 from HIV-1.

24. A process according to claim 23 wherein said oligomeric gp160 has the sequence of residues 33-681 of SEQ ID NO:1.

25. A process according to claim 19 wherein the antigen is a peptide or peptide oligomer.

26. A process according to claim 19 wherein the protein or peptide is recombinantly produced.

27. A process according to claim 19, wherein said vaccine composition is formed by
(a) bonding the hydrophobic material to said protein or peptide to form a hydrophobic-hydrophilic compound; and
(b) admixing said compound with said proteosomes, bioadhesive nanoemulsions, or both such that said antigen is complexed with said proteosomes or nanoemulsion.

28. A process according to claim 27 wherein said admixing step is performed in the presence of a detergent, and is followed by the step of
(c) removing the detergent by dialysis.

29. A process according to claim 27 wherein said admixing step is performed lyophilization.

30. A process for inducing a neutralizing antibody response in a subject against a pathogenic organism resulting in neutralizing antibodies in one or more of vaginal secretions, intestinal secretions, lung secretions and feces, which process comprises administering to said subject by intranasal or respiratory route a vaccine composition according to claim 16.

31. A process according to claim 19 wherein the pathogenic organism is a causative agent of a mucosally-transmitted or sexually transmitted disease.

32. A process according to claim 30, wherein the pathogenic organism is a causative agent of a mucosally transmitted or sexually transmitted disease.
